SEQUENCE LISTING (61 sequences)

SEQ ID NO: 1 - DNA, 37 bp, unidentified
  source 1..37: mol_type = other DNA
atgggccgagacagggaccagtaccagatgtccggac

SEQ ID NO: 2 - DNA, 37 bp, unidentified
  source 1..37: mol_type = other DNA
catcgggtactggtccctgccgatgatatcgtgatgg

SEQ ID NO: 3 - DNA, 82 bp, unidentified
  source 1..82: mol_type = other DNA
ctggtcatcatcctgcctttggtctcgattgnnnnnnnnnnnnnnnnnnnngtttcgagacctgttatatggagggggcaaa

SEQ ID NO: 4 - DNA, 82 bp, unidentified
  source 1..82: mol_type = other DNA
ggattattcataccgtcccaggtctcgattgnnnnnnnnnnnnnnnnnnnngtttcgagaccaatcagccataccacatttg

SEQ ID NO: 5 - DNA, 82 bp, unidentified
  source 1..82: mol_type = other DNA
agagctcgtttagtgaaccgggtctcgattgnnnnnnnnnnnnnnnnnnnngtttcgagaccgatgagtttggacaaaccac

SEQ ID NO: 6 - DNA, 82 bp, unidentified
  source 1..82: mol_type = other DNA
cgttggctacccgtgatattggtctcgattgnnnnnnnnnnnnnnnnnnnngtttcgagaccctattcggctatgactgggc

SEQ ID NO: 7 - DNA, 82 bp, unidentified
  source 1..82: mol_type = other DNA
tgtaaaacgacggccagtggtctcgattgnnnnnnnnnnnnnnnnnnnngtttcgagacccgtaatcatggtcatagctgtt

SEQ ID NO: 8 - DNA, 79 bp, unidentified
  source 1..79: mol_type = other DNA
ggaaacagctatgaccatgggtctcgattgnnnnnnnnnnnnnnnnnnnngtttcgagaccgtcgtgactgggaaaacc

SEQ ID NO: 9 - DNA, 89 bp, unidentified
  source 1..89: mol_type = other DNA
cgactcactatagggagagcggcggtctcgattgnnnnnnnnnnnnnnnnnnnngtttcgagaccctgccatggaaaatcgatgttctt

SEQ ID NO: 10 - DNA, 84 bp, unidentified
  source 1..84: mol_type = other DNA
tcctccgcttattgatatgcggtctcgattgnnnnnnnnnnnnnnnnnnnngtttcgagaccccttgttacgacttttacttcc

SEQ ID NO: 11 - DNA, 82 bp, unidentified
  source 1..82: mol_type = other DNA
gcgagagtagggaactgcggtctcgattgnnnnnnnnnnnnnnnnnnnngtttcgagaccgtgtctcaaaatctctgatgtt

SEQ ID NO: 12 - DNA, 80 bp, unidentified
  variation 32..51: note = N= A, G, T or C
  source 1..80: mol_type = other DNA
ccaagctatttaggtgacacggtctcgattgnnnnnnnnnnnnnnnnnnnngtttcgagacctagaaggcacagtcgagg

SEQ ID NO: 13 - DNA, 20 bp, unidentified
  source 1..20: mol_type = other DNA
ctggtcatcatcctgccttt

SEQ ID NO: 14 - DNA, 20 bp, unidentified
  source 1..20: mol_type = other DNA
tttgccccctccatataaca

SEQ ID NO: 15 - DNA, 20 bp, unidentified
  source 1..20: mol_type = other DNA
ggattattcataccgtccca

SEQ ID NO: 16 - DNA, 20 bp, unidentified
  source 1..20: mol_type = other DNA
caaatgtggtatggctgatt

SEQ ID NO: 17 - DNA, 20 bp, unidentified
  source 1..20: mol_type = other DNA
agagctcgtttagtgaaccg

SEQ ID NO: 18 - DNA, 20 bp, unidentified
  source 1..20: mol_type = other DNA
gtggtttgtccaaactcatc

SEQ ID NO: 19 - DNA, 20 bp, unidentified
  source 1..20: mol_type = other DNA
cgttggctacccgtgatatt

SEQ ID NO: 20 - DNA, 20 bp, unidentified
  source 1..20: mol_type = other DNA
gcccagtcatagccgaatag

SEQ ID NO: 21 - DNA, 18 bp, unidentified
  source 1..18: mol_type = other DNA
tgtaaaacgacggccagt

SEQ ID NO: 22 - DNA, 22 bp, unidentified
  source 1..22: mol_type = other DNA
aacagctatgaccatgattacg

SEQ ID NO: 23 - DNA, 19 bp, unidentified
  source 1..19: mol_type = other DNA
ggaaacagctatgaccatg

SEQ ID NO: 24 - DNA, 18 bp, unidentified
  source 1..18: mol_type = other DNA
ggttttcccagtcacgac

SEQ ID NO: 25 - DNA, 23 bp, unidentified
  source 1..23: mol_type = other DNA
cgactcactatagggagagcggc

SEQ ID NO: 26 - DNA, 24 bp, unidentified
  source 1..24: mol_type = other DNA
aagaacatcgattttccatggcag

SEQ ID NO: 27 - DNA, 20 bp, unidentified
  source 1..20: mol_type = other DNA
tcctccgcttattgatatgc

SEQ ID NO: 28 - DNA, 22 bp, unidentified
  source 1..22: mol_type = other DNA
ggaagtaaaagtcgtaacaagg

SEQ ID NO: 29 - DNA, 18 bp, unidentified
  source 1..18: mol_type = other DNA
gcgagagtagggaactgc

SEQ ID NO: 30 - DNA, 22 bp, unidentified
  source 1..22: mol_type = other DNA
aacatcagagattttgagacac

SEQ ID NO: 31 - DNA, 22 bp, unidentified
  source 1..22: mol_type = other DNA
ccaagctatttaggtgacacgg

SEQ ID NO: 32 - DNA, 20 bp, unidentified
  source 1..20: mol_type = other DNA
cctcgactgtgccttctagg

SEQ ID NO: 33 - DNA, 20 bp, unidentified
  source 1..20: mol_type = other DNA
tactagatcgacgctactag

SEQ ID NO: 34 - DNA, 19 bp, unidentified
  source 1..19: mol_type = other DNA
ccgactcggtgccactttt

SEQ ID NO: 35 - DNA, 21 bp, unidentified
  source 1..21: mol_type = other DNA
cccctgggaatctgaaagaag

SEQ ID NO: 36 - DNA, 19 bp, unidentified
  source 1..19: mol_type = other DNA
ccgactcggtgccactttt

SEQ ID NO: 37 - DNA, 21 bp, unidentified
  source 1..21: mol_type = other DNA
cccctgggaatctgaaagaag

SEQ ID NO: 38 - DNA, 19 bp, unidentified
  source 1..19: mol_type = other DNA
ccgactcggtgccactttt

SEQ ID NO: 39 - DNA, 21 bp, unidentified
  source 1..21: mol_type = other DNA
cccctgggaatctgaaagaag

SEQ ID NO: 40 - DNA, 19 bp, unidentified
  source 1..19: mol_type = other DNA
ccgactcggtgccactttt

SEQ ID NO: 41 - DNA, 24 bp, unidentified
  source 1..24: mol_type = other DNA
attgctctactttctccctcatct

SEQ ID NO: 42 - DNA, 24 bp, unidentified
  source 1..24: mol_type = other DNA
aaacagatgagggagaaagtagag

SEQ ID NO: 43 - DNA, 21 bp, unidentified
  source 1..21: mol_type = other DNA
taaagacaccctccatgttcc

SEQ ID NO: 44 - DNA, 21 bp, unidentified
  source 1..21: mol_type = other DNA
aaattgacaatgaacgttgcc

SEQ ID NO: 45 - DNA, 34 bp, unidentified
  source 1..34: mol_type = other DNA
tagcatctgaatttcataaccaatctcgatacac

SEQ ID NO: 46 - DNA, 21 bp, unidentified
  source 1..21: mol_type = other DNA
tcgttgttgacttccaaatcc

SEQ ID NO: 47 - DNA, 21 bp, unidentified
  source 1..21: mol_type = other DNA
tgaggatcagttgtaccaggg

SEQ ID NO: 48 - DNA, 34 bp, unidentified; identical to SEQ ID NO: 45
  source 1..34: mol_type = other DNA

SEQ ID NO: 49 - DNA, 21 bp, unidentified
  source 1..21: mol_type = other DNA
agcaaaaggtacctgatgacg

SEQ ID NO: 50 - DNA, 21 bp, unidentified
  source 1..21: mol_type = other DNA
cttttcgtcgaggtagtgctg

SEQ ID NO: 51 - DNA, 26 bp, unidentified
  source 1..26: mol_type = other DNA
ataataacgctgcggacatctacatt

SEQ ID NO: 52 - DNA, 31 bp, unidentified
  source 1..31: mol_type = other DNA
caccatggacagatctcaagaacactatgcc

SEQ ID NO: 53 - DNA, 27 bp, unidentified
  source 1..27: mol_type = other DNA
tcacacactttgtatgtttgtgtgacc

SEQ ID NO: 54 - DNA, 30 bp, unidentified
  source 1..30: mol_type = other DNA
caccatggaaataactcaagtaatctatgt

SEQ ID NO: 55 - DNA, 31 bp, unidentified
  source 1..31: mol_type = other DNA
tcatacactatgtatgtttgtgtgaccttcc

SEQ ID NO: 56 - DNA, 31 bp, unidentified
  source 1..31: mol_type = other DNA
caccatgggcatatctcaagtacactattgc

SEQ ID NO: 57 - DNA, 32 bp, unidentified
  source 1..32: mol_type = other DNA
tcataaagtttgtgtttcttcctcaacaggtt

SEQ ID NO: 58 - DNA, 20 bp, unidentified
  source 1..20: mol_type = other DNA
ctctactttctccctcatct

SEQ ID NO: 59 - DNA, 21 bp, unidentified
  source 1..21: mol_type = other DNA
tatcatggaaaactgtcactg

SEQ ID NO: 60 - DNA, 11 bp, unidentified
  source 1..11: mol_type = other DNA
ggtctcgattg

SEQ ID NO: 61 - DNA, 11 bp, unidentified
  source 1..11: mol_type = other DNA
gtttcgagacc

(19) United States
(12) Patent Application Publication
(10) Pub. No.: US 2025/0270573 A1
(43) Pub. Date: Aug. 28, 2025

(54) SYSTEMS AND METHODS FOR GENOME-SCALE TARGETING OF FUNCTIONAL REDUNDANCY IN PLANTS

(71) Applicant: Ramot at Tel-Aviv University Ltd., Tel Aviv (IL)

(72) Inventors: Eilon SHANI, Tel Aviv (IL); Itay MAYROSE, Tel Aviv (IL)

(21) Appl. No.: 18/855,815

(22) PCT Filed: Apr. 3, 2023
(86) PCT No.: PCT/IL2023/050351

§ 371 Date: Oct. 10, 2024

Related U.S. Application Data
(60) Provisional application No. 63/329,506, filed on Apr. 11, 2022.

(51) Int. Cl. C12N 15/82; C12N 15/10; C12N 15/11

(52) U.S. Cl. CPC C12N 15/8213; C12N 15/1093; C12N 15/11; C12N 2310/20

(57) ABSTRACT

The present invention relates to compositions and methods for overcoming functional redundancy in plants, particularly to methods for knocking-out and identifying multiple genes underlying a certain phenotype, utilizing multi-targeted genome-scale Clustered Regularly Interspaced Short Palindromic Repeats (CRISPR) applications.

### FIELD OF THE INVENTION

The present invention relates to compositions and methods for overcoming genetic functional redundancy in plants, particularly to methods for knocking-out and identifying multiple genes underlying a certain phenotype, utilizing multi-targeted genome-scale Clustered Regularly Interspaced Short Palindromic Repeats (CRISPR) applications.

### BACKGROUND OF THE INVENTION

Plant genomics and breeding programs rely on genetic variation, be it natural, induced, or introduced. Genetic variation has been expanded over the years by introducing natural variation and by creating random mutagenized lines by treatment with physical (e.g., radiation), chemical (e.g., ethyl methanesulfonate) or biological (e.g., T-DNA insertion or gene silencing) mutagens. These approaches have greatly facilitated and accelerated progress in plant functional genomics and breeding programs over the past several decades.

Comprehensive genetic studies and large-scale genome sequencing projects have shown that it is challenging to alter many phenotypes due to the genetic redundancy in plants. Local and global gene duplications over the course of plant evolution have resulted in large gene families of similar sequences and partially overlapping functions. On average, 64.5% of plant genes are paralogous, ranging from 45.5% in the moss Physcomitrella patens to 84.4% in the apple Malus domestica. Given that ancient and/or fast-evolving paralogs are not easily detected due to sequence divergence, these percentages are likely underestimated. In Arabidopsis thaliana, the paralog gene content is around 63%. In addition, 22,020 Arabidopsis genes, representing 78% of all protein-coding genes, belong to families with at least two members. It is speculated that single-copy genes are likely to be involved in the maintenance of genome integrity and organelle function, whereas multi-copy genes encode proteins involved in signaling, transport, and metabolism. Therefore, mutating multiple members of a gene set is required to uncover “hidden” phenotypes in many cases. As of 2014, only about 8% of Arabidopsis genes were reported to have a loss-of-function mutant phenotype, and about 1.5% of Arabidopsis genes exhibited an observable phenotype only when disrupted in combination with a redundant paralog.

Forward-genetics is an approach for the determination of the genomic basis of an observed phenotype. Means of creating random mutations for forward-genetics (e.g., alkylating agents and T-DNA lines) cannot simultaneously target multiple genes belonging to one group in a single mutant line and thus cannot overcome the limitations of genetic redundancy, especially when the genes of interest are genetically linked. In recent years, significant progress has been made using genome-scale RNA interference methods and artificial microRNA (amiRNA) collections; however, these methods generally reduce gene expression rather than causing complete knockout phenotypes and do not work well in several important crops.

Recently, CRISPR/Cas systems, involving CRISPR repeat-spacer arrays and Cas proteins, have been used to build large knockout mutant libraries for forward-genetic screens and for analysis of gene functions and regulation in the genomic context. This system represents a massive breakthrough for generating targeted mutations both in terms of simplicity and efficiency. Studies carried in the past few years have demonstrated the feasibility of CRISPR-based single-gene knockout collections in rice and tomato.

Hyams et al. to inventors of the present invention and co-workers disclose optimal sgRNA design for editing multiple members of a gene family using the CRISPR System (J. Mol Biol (2018) 430, 2184-2195).

However, thus far, CRISPR/Cas has not been used on a genome-scale level to target multiple potentially redundant genes in eukaryotes, including plants. There is a need in the field of plant trait optimization and breeding programs for an efficient, high throughput technologies for elucidating plant gene functions.

### SUMMARY OF THE INVENTION

The present invention relates to the development and validation of a Multi-Knock, “next-generation” genetic approach preferably to be used in plants, that combines forward-genetics with dynamically targeted genome-scale CRISPR/Cas tools to address the problem of masked phenotypic variation due to genetic functional redundancy, and characterize most or all the members of a multi-gene set. The multi-gene set can represent a multi-gene family, multiple genes involved in a certain pathway, or a combination thereof.

Unexpectedly, the inventors of the present invention succeeded in applying a genome-wide, forward genetic screening method in planta. The method, designed to overcome the redundancy challenge in plant, was able to identify multiple genes underlying a specific phenotype without the need of using, for example, in vitro digestion assays to validate knockout activity.

The multi-targeted CRISPR libraries described herein comprise different sgRNAs targeting plurality of gene members within a gene set.

In some embodiments, the multi-targeted CRISPR libraries described herein comprises two or more different sgRNAs targeting the same gene or genes within a gene set. It is now disclosed that this multiplex approach, i.e., two or more sgRNAs targeting the same genes, enables an improved knock-out efficiency of the targeted gene members. The different sgRNA in some embodiments are present in the same construct.

The present invention is based, in part, on the unexpected results demonstrating the ability of the systems and methods of the invention to expose redundant genes contributing to a single phenotype at a genome-scale level. The phenotype may be, among others, an agricultural trait, a phenotype of a molecular pathway, or a phenotype of a functional pathway. Identifying most or all the genes contributing to the phenotype is of significant importance in plant breeding programs targeted at obtaining stable lines characterized by a certain phenotype. The systems and methods of the present invention provides for a genome-wide knockout of multiple members of a specific gene set, over multiple gene sets in the genome, utilizing novel sgRNAs within a CRISPR library which is subsequently transformed into plants, enabling the production and exposure of a plurality of phenotypes which cannot be achieved via traditional breeding methods. In the course of the research of the present invention, the inventors generated an improved genome-editing efficient intronized Cas9 vector (or other Cas9 vectors), into which a total of newly designed and synthesized 59,129 multi-targeted sgRNAs in 10 libraries targeting 16,152 genes in Arabidopsis (˜74% of all protein-coding genes that belong to families), have been cloned. In some embodiments of the invention, 5,635 sgRNAs targeting 1,327 members of the TRANSPORTERS (TRP) family in Arabidopsis were cloned into four different Cas9 vectors generating independent CRISPR libraries, wherein each sgRNA was designed to target closely homologous genes within sub-clades in transporter families. Based on the methods of the invention, using a newly designed forward-genetic screen which employs over 3,500 CRISPR lines targeting the plant transportome, novel redundant transporters in Arabidopsis have been identified, demonstrating the validity of the systems and methods of the invention. Among many others, the hitherto unknown genes PUP7, PUP21, and PUP8, encoding cytokinin transporters, have been revealed. Further disclosed herein is that PUP8 localizes to the plasma membrane and that PUP7 and PUP21 are localized to the tonoplast. Together, these proteins regulate meristem size, phyllotaxis, and plant growth. The Multi-Knock technology of the present invention is a powerful and efficient tool that can be used to uncover hidden phenotypic variations. Its use may accelerate plant breeding programs and facilitate plant functional genomics studies.

In other embodiments, multi-targeted CRISPR libraries were generated for tomato. A total of 15,804 sgRNAs targeting 13,590 genes were designed and synthesized. Each sgRNA targets multiple genes. The large library was divided into several sub-libraries targeting specific gene sets, several of which were cloned and introduced into plants, generating over a hundred independent tomato lines. In yet other embodiments, multi-targeted CRISPR libraries were generated for rice: a total of 634 sgRNA targeting 405 genes were designed and synthesized. Each sgRNA targets multiple genes. The library was divided into two sub-libraries targeting specific gene sets. Each gene set comprises 300-500 sgRNA targeting 150-400 genes. The libraries were cloned and introduced into plants, generating independent 1,000 rice lines having CRISPR systems with different sgRNAs.

Accordingly, disclosed herein are methods for sgRNA oligonucleotide library design followed by construction of a CRISPR library and its subsequent transformation into plants, allowing for screening of the desired phenotype; whereby said phenotype reflects the targeted knockout of multiple gene members belonging to the same gene set of a gene family or genes involved in a pathway. The design of multiple sgRNA may be based on in silico genomic data, or on genetic information based on genomic analysis of plant genetic material. The genomic data can include DNA and/or RNA sequence data and the analysis can be performed by any method as is known in the Art, including next-generation sequencing (NGS), RNA-sequencing (RNA-seq) and other transcriptomics methods. In certain embodiments, the genomic data of the target plant is filtered so as to exclude mitochondrial, chloroplast and singleton genes. In certain exemplary embodiments, the genetic data is then partitioned into clusters using, for example, the CRISPys computational algorithm (Hyams et al., ibid), which employs combinatorics and graph theory to design the optimal guide RNAs that could most efficiently target the family of genes.

According to certain aspects, the present invention provides a method for identifying multiple members within at least one gene set underlying a phenotype, the method comprising:
(i) clustering coding sequences within genetic data of a plant species to sequence clusters, each cluster representing a gene set;
(ii) producing a CRISPR library comprising a plurality of polynucleotides, wherein each polynucleotide encodes one or more unique sgRNAs, wherein each of the sgRNA targets a plurality of gene members comprised within the gene set;
(iii) transforming the library into a plurality of plants, thereby producing a plant population wherein each plant of the population comprises at least one sgRNA targeting multiple gene members;
(iv) screening the plant population for at least one selected phenotype;
(v) selecting at least one plant showing the at least one selected phenotype; and
(vi) identifying in the selected plant the at least one sgRNA targeting the multiple-gene members;
thereby identifying said multiple gene members underlying said selected phenotype.

According to some embodiments, at least two of the unique sgRNAs target a single gene member.

According to certain embodiments, at least two of the unique sgRNAs target at least two same gene members out of a plurality of gene members targeted by the at least two unique sgRNAs.

According to some embodiments, at least two of the unique sgRNAs target the same plurality of gene members.

According to some embodiments, the polynucleotides encoding the at least two of the unique sgRNAs are present in a single construct.

According to some embodiments, the library comprises at least one polynucleotide encoding for two different sgRNAs targeting the same gene members.

According to certain embodiments, the gene set comprises genes of a single gene family. According to these embodiments, clustering the coding sequences comprises clustering coding sequences encoding polypeptides, the polypeptides having at least 30% sequence identity.

According to theses embodiments, clustering the coding sequences comprises clustering coding sequences encoding polypeptides, the polypeptides having at least 40%, 50%, 60%, 70%, or 80% sequence identity. Each possibility represents a separate embodiment of the invention.

According to some embodiments, the method comprises a step of further sub-grouping the gene set based on their sequence similarity.

According to certain embodiments, the gene set comprises genes forming part of a functional or molecular pathway. According to these embodiments, clustering the coding sequences is based on the functional or molecular pathway.

In certain embodiments the genetic data are selected from the group consisting of genomic sequencing data, RNA sequencing data, spatial transcriptomics, ribosome profiling, proteomics and protein-protein interactomics data. Each possibility represents a separate embodiment of the present invention.

According to certain exemplary embodiments, the RNA sequencing data are selected from total RNA-seq and transcriptomics. Each possibility represents a separate embodiment of the present invention.

In some embodiments, producing the CRISPR library comprises designing the plurality of sgRNAs following an analysis of the genetic data of the plant species, the analysis comprising filtering out mitochondrial, chloroplast and/or singleton genes.

In certain embodiments, the plurality of sgRNAs is designed using a computational algorithm determining the probability that multiple genomic targets are cleaved by a given sgRNA. According to certain embodiments, the algorithm evaluates all possible sgRNA target sites within the exonic regions on both DNA strands, across all gene family members, and ranks those target sites based on at least one of cleavage probability, position within the gene, off target effects and any combination thereof. According to certain embodiments, the algorithm evaluates all possible sgRNA target sites within promoters, introns, or untranslated regions (UTRs). According to additional embodiments, the algorithm evaluates all possible sgRNA target sites for targeting tandem genes (genetically linked genes) by creating large deletion with one or more sgRNAs.

In certain embodiments, sgRNA molecule or molecules that target a single gene underlying a phenotype are removed.

In additional embodiments, sgRNAs are classified according to a given functional classification, depending on the desired interest in the genetic screen or breeding program.

According to certain exemplary embodiments, sgRNAs are classified to form a plurality of sub-functional libraries according to the protein function(s) of the sgRNA putative target genes within a gene set.

According to certain embodiments, the method comprises producing a plurality of libraries, each library comprising a plurality of polynucleotides, wherein each polynucleotide encoding one or more unique sgRNAs targeting a plurality of gene members comprised within a gene set, wherein each library comprises a different gene set.

According to certain embodiments, the method comprises producing from 2 to at least 5, at least 10, at least 100, at least 200, at least 500 or more libraries.

According to certain embodiments, the plurality of libraries comprises from 2 to at least 100, at least 500, at least 1,000, at least 5,000, at least 10,000 or more libraries. According to these embodiments, the plurality of libraries may be designated as “large” or “mega” library.

In some embodiments the large-library and/or each of the sub-libraries is targeting genes encoding a gene set selected from the group consisting of: transporters; protein kinases; protein phosphatases; receptors, and their ligands; transcription factors; protein binding small molecules; proteins that form or interact with protein complexes including stabilizing factors; hydrolytic enzymes, excluding protein phosphatases; catalytically active proteins, mainly enzymes; metabolic enzymes and enzymes that catalyze transfer reactions; gene set expressed within a plant organ; genes involved in resistance to biotic stress; gene involved in resistance to abiotic stress; proteins of unknown function, and the like. Each possibility represents a separate embodiment of the present invention.

In additional embodiments, adaptor nucleotides, unique to each gene set, are to facilitate amplification of each library in the plurality of libraries.

According to certain embodiments, the CRISPR library further comprises a nucleic acid sequence encoding an RNA-guided DNA endonuclease enzyme. According to some embodiments, the endonuclease is selected from the group consisting of caspase 9 (Cas9), Cpf1, or other Cas proteins. According to certain exemplary embodiments, the endonuclease is Cas9.

The CRISPR libraries may be produced using any method as is known in the art. According to certain embodiments, the polynucleotide encoding the sgRNA and the nucleic acid sequence encoding the RNA-guided DNA endonuclease enzyme, particularly Cas9, are present within a single vector.

According to certain embodiments, the polynucleotide encoding ethe sgRNA molecules and the nucleic acid sequence encoding the RNA-guided DNA endonuclease enzyme each is present on a separate vector.

According to yet further embodiment, each of the vectors comprising the polynucleotide encoding the one or more sgRNA molecules and the vector comprising the polynucleotide encoding the RNA-guided DNA endonuclease enzyme is transformed to a separate plant. According to these embodiments, the method further comprises crossing the plants to form a progeny comprising both, the polynucleotide encoding the sgRNA and the polynucleotide encoding the RNA-guided DNA endonuclease enzyme, particularly Cas9.

According to yet additional embodiments, the vector comprising the polynucleotide encoding the one or more sgRNA is transformed to a plant comprising an RNA-guided DNA endonuclease enzyme, particularly Cas9.

According to certain exemplary embodiments, a polynucleotide encoding the one or more sgRNAs designed to target a plurality of genes is cloned into a single intronized zCas9 vector comprising a number of introns integrated into the maize codon-optimized Cas9.

According to certain embodiments, the one or more unique sgRNAs comprise at least 10, at least 50, at least 100, at least 200, at least 300, at least 400, at least 500, at least 600, at least 700, at least 800, at least 900, at least 1000, or more, sgRNAs. According to certain exemplary embodiments the one or more unique sgRNAs comprises from about 20 to about 10,000 sgRNAs.

In some embodiments, the cloned vectors are transformed into bacteria and the vector identity is validated using bacterial selection medium followed by plasmid DNA purification, amplification, and deep sequencing.

In additional embodiments, the library or the plurality of libraries is transformed into a plurality of plants to form a plurality of transformed plants, each transformed plant expressing at least one sgRNA, each sgRNA targeting multiple members of a gene set. It is to be understood that according to theses embodiments, the plurality of libraries comprises sgRNAs targeting a plurality of gene sets.

The plants to be used in the method of the present invention can be wild type plant as well as plant cultivars, the later can be hybrid lines or inbred lines. According to certain embodiments, the plants are monocot plants. According to other embodiments, the plants are dicot plants. According to certain exemplary embodiments, the plants to be transformed are not genetically modified. According to certain embodiments, the plants to be transformed are of the same species.

According to certain embodiments, screening the plant population for the selected phenotype comprises subjecting said plant population to at least one abiotic stress. According to certain embodiments, the abiotic stress is selected from the group consisting of heat stress, salt stress and drought stress. Each possibility represents a separate embodiment of the present invention.

Any phenotype can be selected for screening the transformed plant population. According to certain embodiments, the phenotype is an agricultural trait. Any agricultural trait can serve as the selected phenotype. According to some embodiments, the agricultural trait is selected from the group consisting of yield, harvest index, growth rate, biomass, plant vigor, root system, leaf color, rosette size, plant height, flowering time, photosynthetic capacity, nitrogen use efficiency, biotic stress resistance, abiotic stress resistance and any combination thereof. Each possibility represents a separate embodiment of the present invention.

According to certain embodiments, the phenotype is linked to an artificially-introduced trait. According to certain embodiments, the phenotype is attributed to a suppressor or enhancer linked to a genetic manipulation intentionally introduced into the plants, including, for example, plants holding a phenotype caused by a mutation or over-expression for suppressor/enhancer screen. Similarly, according to certain embodiments, the phenotype is attributed to a suppressor or enhancer linked to a genetic manipulation that allows expression of a visible marker genes (e.g. fluorescent proteins (GFP), enzyme reporters (GUS or LUC) and resistance-conferring genes).

According to an additional aspect the present invention provides a construct comprising a plurality of polynucleotides each encoding a unique sgRNA targeting the same gene members within a gene set as described herein.

According to some embodiments, each polynucleotide encodes two different sgRNAs targeting the same gene members within a gene set as described herein.

According to some embodiments, the construct further comprises means for CRISPR activity. According to certain embodiments, the construct comprises a nucleic acid encoding an RNA-guided DNA endonuclease as described herein.

According to an additional aspect, the present invention provides a library comprising a plurality of constructs, each construct comprises a pair of polynucleotides each encoding a different sgRNA, the sgRNAs targeting the same gene members within a gene set as described herein.

According to certain additional aspects, the present invention provides a library for screening multiple members within at least one gene set, the library comprising a plurality of vectors, each vector comprising one or a plurality of polynucleotides encoding one or more unique sgRNAs, wherein each sgRNA is targeted to a plurality of genes, wherein the plurality of genes comprises members of a gene set as described herein.

According to certain exemplary embodiments, the vector further comprises at least one regulatory element operably linked to each polynucleotide encoding sgRNA.

According to certain embodiments, the library is a CRISPR library further comprising a nucleic acid sequence encoding an RNA-guided DNA endonuclease enzyme.

According to certain embodiments, the endonuclease is selected from the group consisting of caspase 9 (Cas9), Cpf1, or other Cas proteins. According to certain exemplary embodiments, the endonuclease is Cas9.

According to certain exemplary embodiments, each of the vectors of the library comprises at least one of the polynucleotides encoding sgRNAs and a nucleic acid sequence encoding the endonuclease. According to certain exemplary embodiments, the endonuclease is Cas9.

According to further certain exemplary embodiments, each vector further comprises at least one selectable marker. The term “selectable marker” refers to a gene which encodes an enzyme having an activity that confers resistance to an antibiotic or drug upon the cell in which the selectable marker is expressed, or which confers expression of a trait which can be detected (e.g., luminescence or fluorescence).

According to certain exemplary embodiments, the marker is a “positive” marker. Examples of positive selectable markers include the neomycin phosphotrasferase (NPTII) gene that confers resistance to G418 and to kanamycin, the bacterial hygromycin phosphotransferase gene (hyg), which confers resistance to the antibiotic hygromycin, and Phosphinothricin (PPT) (or Basta) that blocks nitrogen assimilation.

According to certain embodiments, the plurality of vectors comprises sgRNAs targeting a plurality of gene sets of an entire genome of a plant species. The gene sets are multi-member gene sets as described herein.

It is to be understood that any combination of each of the aspects and the embodiments disclosed herein is explicitly encompassed within the disclosure of the present invention.

Other objects, features and advantages of the present invention will become clear from the following description and drawings.

### BRIEF DESCRIPTION OF THE FIGURES

FIG. 1 depicts an overview of the Multi-Knock, genome-scale, multi-targeted CRISPR platform. Stage 1: Multi-targeted sgRNAs were designed to target multiple genes (coding sequences) from the same gene family. The Arabidopsis genome was clustered into gene families and multiple sgRNAs were designed to target each node using the CRISPys algorithm. Stages 2 and 3: sgRNA sub-library sequences were synthesized, amplified, and cloned into CRISPR/Cas9 vectors. Stage 4: The library was introduced into Agrobacterium and transformed into Arabidopsis to generate stable lines. Each plant expresses a single sgRNA, targeting a clade of 2 to 10 genes from the same family. Stage 5: A phenotypic forward genetic screen was conducted. Candidate lines were genotyped for sgRNAs and targets.

FIG. 2 shows an overview of sgRNA design strategy for gene families. For each gene family, the multiple alignments of the respective protein sequences are computed. P stands for protein, and letters indicate amino acids. A phylogenetic tree is constructed based on the sequence similarity of the protein sequences. Optimal sgRNAs for each subgroup of genes, which are induced by internal nodes in the tree (marked by lowercase letters a-c), are then designed. For each subfamily of genes, and illustrated here for node a, all potential CRISPR target sites are extracted. In this case, the subfamily induced by node a includes two genes (g_2 and g_4, encoding for proteins P_2 and P_4, respectively). Typically, each gene contains dozens of possible targets. For simplicity, only five targets are presented. Nucleotide positions that are identical or polymorphic sites are differentiated by different grayscales colors. Next, a tree of the target sites is constructed based on sequence similarity among the targets while accounting for CRISPR-specific characteristics. sgRNA candidates are constructed for each internal node, where all combinations of the polymorphic sites are considered, and the ones with the highest editing efficacy to target the considered subgroup of genes are chosen. For simplicity, only a few candidates (denoted by si) are shown for each internal node. Assuming that the cutoff of the number of polymorphic sites k is 4, the search of sgRNA candidates stops at node z. In practice, k was set to 12 polymorphic sites.

FIGS. 3A-3F illustrates the design and construction of multi-targeted genome-scale sgRNA. FIG. 3A—Schematic illustration of the computational workflow used to design the Multi-Knock sgRNA library. A filtering process yielded a selection of 59,129 sgRNAs targeting 16,152 genes (˜74% of all coding genes belonging to families). Abbreviations: Mt-genes, mitochondrial genes; Cp-genes, chloroplast genes; Singletons, genes that do not belong to a family. FIG. 3B—Histogram showing the number of genes targeted by individual sgRNAs. FIG. 3B—Representative sgRNA-target network in the CRISPR library. Genes are targeted by multiple sgRNAs, and sgRNAs target multiple genes. FIG. 3D—Total number of sgRNAs and target genes in each functional sub-library. FIG. 3E-3F—Deep-sequencing data of sgRNAs in individual sub-libraries. Columns indicate the distribution of sgRNAs. Coverage is indicated for each group.

FIGS. 4A-4C illustrate the transportome-specific Multi-Knock screen. FIG. 4A—To create independent sub-libraries, 5,635 sgRNAs, each targeting 2 to 10 transporters from the same family, were amplified and cloned into four different Cas9 vectors to create pRPS5A:Cas9 (OLE: CITRIN), pUBI:Cas9, pEC:Cas9, and pRPS5A:zCas9i sub-libraries. Graphs show coverage and frequency based on next-generation sequencing of the four sub-libraries. The four libraries were transformed into Col-0 plants yielding 3,500 transgenic T1 plants. FIG. 4B—Photographs show representative phenotypes of TRP Multi-Knock proof-of-concept lines. From top to bottom are Col-0 and plant expressing sgRNA targeting toc120 and toc132 (scale bar=2 cm), Col-0 and plant expressing sgRNA targeting mex1 and mex11 (scale bar=1 cm), and control DR5: VENUS plant and the T1 plant harboring sgRNA targeting bor1 and bor2 (scale bar=4 cm). Chromatograms show the types of mutations. Arrows indicate the mismatches between sgRNA and target sequence. PAM is marked with a black underline. FIG. 4C—Images show lines with abnormal phenotypes that had not previously been described: from top to bottom adjacent to Col-O control are plants expressing sgRNA targeting clc-a and clc-b (scale bar=2 cm), vha-d1 and vhad-2 (scale bars=2 cm), and pup8 and pup21 (scale bar=1 cm). Chromatograms show the type of mutations. Arrows indicate the mismatches between sgRNA and target sequence. PAM is marked with a black underline.

FIGS. 5A-5F illustrate the redundant regulation of phyllotaxis by PUP7, PUP8, and PUP21. FIG. 5A—Phylogenetic tree of Arabidopsis PUP family based on amino acid sequences. Gray dots indicate proteins coded by putative CR7/8/21 target genes. FIG. 5B—Chromatograms showing the types of mutations in the CR7/8/21 line as identified by sequencing. CR7/8/21 stands for CRISPR triple mutant PUP7/8/21. PAM is underlined in black; the 20-bp gRNA is underlined. FIG. 5C—Phyllotaxis patterns in inflorescences stem of wild-type (Col-0), single T-DNA insertion mutants and CR7/8/21. Scale bar=2 cm. FIG. 5D—Silique divergence angle distribution in inflorescences of Col-0, pup single mutants, and CR7/8/21. P-value, n number and standard error (sd) are indicated for each analysis. P-value was extracted using Fligner-Killeen test for equality of variance. FIG. 5E—Phyllotaxis patterns in inflorescence stem of control (TCS: VENUS) and amiRNA7/8/21 mutant. amiRNA7/8/21 stands for amiRNA triple PUP7/8/21 knockdown. Scale bar=2 cm. FIG. 5F—Distribution of divergence angle frequencies between successive siliques in control and amiRNA7/8/21 stems. p value Fligner-Killeen test for equality of variance is indicated for each analysis.

FIG. 6 shows the selection of Cas9-free in the pRPS5A:Cas9 OLE:CITRINE T2 generation. Bright signal in seeds indicates for OLE:CITRINE. Examples of Cas9-free seeds, which do not produce the bright fluorescence, are marked by arrows. Scale bar=1 mm.

FIGS. 7A-7D show multi-targeted genome-scale sgRNA design in tomato. FIG. 7A—Illustration of the computational workflow used to design the genome-wide CRISPR screen for phenotypes governed by functional redundancy. The computational design process yielded 15,804 sgRNAs targeting 13,590 genes (˜50% of all coding genes). Mt, mitochondrial; Cp, chloroplast; Singletons, genes without any family members. FIG. 7B—Histogram showing the number of genes targeted by individual sgRNAs for the entire CRISPR library. FIG. 7C—Example of a typical sgRNA-target network in the CRISPR library. The CRISPR sub-libraries are cloned separately to allow flexibility in the pUBQ4: CAS9 vector, which has high Cas9 activity in tomato. FIG. 7D—The tomato genome-scale sgRNA library was divided into 10 sub-libraries. The illustration shows the number of sgRNAs and the number of genes for each sub-library.

FIGS. 8A-8B show the construction of transportome-specific multi-targeted tomato CRISPR library. Sub-library 1, which includes 450 sgRNAs, was amplified and cloned into UBQ4: CAS9 (FIG. 8A). Next-generation sequencing was used to evaluate sgRNA coverage (100%) and frequency (FIG. 8B).

FIGS. 9A-9C show multi-Crop sgRNA transformation into tomato. FIG. 9A—Tomato tissue culture Multi-Crop transformation. FIG. 9B—T0 lines growing in the greenhouse at TAU. FIG. 9C—30 independent T1 lines were grown in controlled growth rooms with and without NaCl (120 mM) treatment. n=10. Shown are representative images of a larger experiment.

FIGS. 10A-10C show the validation of sgRNA integration in tomato plants. FIG. 10A—PCR genotyping of 10 independent T0 lines showing the expected sgRNA band (for 9 out of 10 lines). N.C stands for negative control. FIG. 10B—sgRNA sequencing chromatograms reveal the putative target genes. FIG. 10C—PCR genotyping of 4 T1 plants from line 8 showing the expected sgRNA band. N.C stands for negative control.

FIG. 11 shows construction of Multi-Knock transportome-specific rice CRISPR library. The library includes 634 sgRNAs that target 405 rice transporters. Next-generation sequencing was used to evaluate sgRNA coverage (99.84%) and frequency.

FIGS. 12A-12B show the validation of sgRNA integration in T1 rice plants. FIG. 12A—PCR genotyping of 4 independent T1 lines showing the expected sgRNA band. N.C stands for negative control. PC stands for positive control. A and B in each line stands for different plants within the line. FIG. 12B—sgRNA sequencing chromatograms for the independent lines reveal the putative target genes.

### DETAILED DESCRIPTION OF THE INVENTION

The present invention discloses compositions and methods for performing targeted knock-out gene modification of multiple members of at least one unique coding gene set in plants. Specific small guide RNAs are designed within a CRISPR system, which in turn is transformed into the target plants, thereby conducting functionality based genetic modification which overcomes genetic redundancy in plants.

Genetic manipulation of plants has revolutionized plant breeding and made possible selective adaptation of numerous plant species according to any number of preferences, from cultivation specifications to nutritional makeup. Traditional breeding techniques have been limited by the natural genetic profile of the desired plant species, with limited ability to (a) affect a targeted perturbation of certain genes in an attempt to create a pre-designed phenotype or to (b) conduct an analytical and precise examination of these genes. The advent of new molecular biology methods and advanced genetic modification techniques has led to profound improvements in this field.

### Definitions

As used herein, the term “genetic redundancy” refers to the existence of multiple different genes performing the same or similar biological function, and that inactivation of only one, or even several of these genes but not all, has little to no effect on the phenotype.

As used herein, the term “a plurality” refers “at least two”, typically more than two.

As used herein, the term “gene set” refers to a plurality of genes sharing certain structural homology or to a plurality of genes participating in a pathway. According to certain embodiments, the pathway is a functional pathway. According to certain embodiments, the pathway is a molecular pathway.

The term “gene family” refers to a group of related genes that share a common ancestor. Members of gene families may be paralogs or orthologs. Gene paralogs are genes with similar sequences from within the same species while gene orthologs are genes with similar sequences in different species. According to certain exemplary embodiments, gene families according to the teachings of the present invention comprise gene paralogs.

The term “library” as used herein refers to a collection of similar sized DNA fragments, a collection that includes several different items. “Library” or “sub-library” are interchangeable and depend on the context. The term “CRISPR library” are used herein to describe a collection of constructs comprising polynucleotides encoding sgRNAs and optionally, additional means for CRISPR such as nucleic acids encoding an RNA-guided DNA endonuclease enzyme.

The term “interactomics” as used herein refers to a discipline at the intersection of bioinformatics and biology that deals with studying both the interactions and the consequences of those interactions between and among proteins, and other molecules within a cell.

The term “Transportome” as used herein refers to all membrane transporters and proteinaceous channels that govern influx and efflux of ions in a cell.

As used herein, the phrase “generating targeted mutations” relates to the commonly known in the art concepts of genetic manipulation/modification/engineering, as defined by altering an organism's genome by insertion, deletion, or alteration of genetic material, as evidenced by observable and measurable changes to the organism's phenotype and genetic expression. To confirm the creation of such a mutagenized line, it is common in the art to employ various sequencing techniques—a well-known methodology utilized to ascertain the nucleic acid sequence of an organism's genome or sgRNA inserts.

Clustered regularly interspaced short palindromic repeats (CRISPR)/Cas systems are known in the art and can be engineered for directed genome editing. Cas genes encode RNA-guided DNA endonuclease enzymes capable of introducing a double strand break in a double helical nucleic acid sequence. The Cas enzyme can be directed to make the double stranded break at a target site within a gene using the single guide RNA (sgRNA) and tracer cellular machinery.

As used herein, the terms “single guide RNA”, “sgRNA” and “gRNA” are used herein interchangeably and refer to a piece of RNA that function as guides for RNA- or DNA-targeting enzymes, which they form complexes with. The targeting specificity of the CRISPR/Cas system is determined by a short sequence (e.g., 20-nt) at the 5′ end of the gRNA. The desired target sequence must precede the protospacer adjacent motif (PAM). After base pairing of the gRNA to the target, Cas mediates a double strand break about 3-nucleotides (nt) upstream of PAM.

A Cas enzyme can be from any appropriate species (e.g., an archaea or bacterial species). For example, a Cas enzyme can be from Streptococcus pyogenes, Pseudomonas aeruginosa, or Escherichia coli. In some cases, a Cas enzyme can be a type I (e.g., type IA, IB, IC, ID, IE, or IF), type II (e.g., IIA, IIB, or IIC), or type III (e.g., IIIA or IIIB) Cas enzyme. The encoded Cas enzyme can be any appropriate homolog or Cas fragment in which the enzymatic function (i.e., the ability to introduce a sequence-specific double strand break in a double helical nucleic acid sequence) is retained. In some embodiments, a Cas enzyme is a Streptococcus pyogenes Cas9 enzyme. In some cases, a Cas enzyme can be codon optimized for expression in particular cells, such as dicot or monocot plant cells. The Cas enzyme can further be a protospacer-adjacent motif (PAM) edited variant, including, for example, the Cas9 enzyme variants SpG and SpRY. A Cas-expressing transgene can include a Cas gene from any appropriate species (e.g., an archaea or bacterial species).

The CRISPys computational algorithm is aimed at designing the optimal guide RNAs that could potentially target multiple members of a given gene set. The algorithm is based on the following steps. First, the algorithm detects all potential targets located within the input gene set. Second, it clusters all potential targets into a hierarchical tree structure that specifies the similarity among them. Then, guide RNAs are computed in the internal nodes of the tree by embedding mismatches where needed. Fourth, the algorithm, identifies the guide RNAs whose propensity to edit the induced targets is maximized. The algorithm can either identify the single guide RNA that could best target the input gene set, or compute multiple guide RNAs that collectively target the entire gene set with highest efficiency. For each of these options, the algorithm makes use of a pre-computed scoring function that specifies the targeting efficiency of a given sgRNA to a given genomic site.

According to certain aspects, the present invention provides a method for identifying multiple members within at least one gene set associated with a certain phenotype, the method comprising: clustering coding sequences within genetic data of a genome of a plant species to sequence clusters, each cluster representing a gene set comprising a plurality of gene members, and selecting at least one gene set; producing a plurality of CRISPR libraries, each library comprising a plurality of polynucleotides encoding unique sgRNAs targeting a plurality of gene members comprised in the gene set; transforming each of the libraries into a plurality of plants, thereby producing a plant population wherein each plant of the population comprises one or more sgRNA targeting multiple gene members of said at least one gene set; screening the plant population for at least one selected phenotype; selecting plants showing the at least one selected phenotype; and identifying in the selected plants the at least one sgRNA targeting the multiple-gene members; thereby identifying said multiple gene members of said at least one gene set associated with said selected phenotype.

According to yet additional aspects, the present invention provides a library for screening multiple members within at least one gene set, the library comprising a plurality of vectors, from 10 to several thousands, each vector comprising one or more polynucleotides each encoding one or more unique sgRNA, wherein each sgRNA is targeted to a plurality of genes, wherein the plurality of genes are members of a gene set. According to certain exemplary embodiments, the vector further comprises at least one regulatory element operably linked to each sgRNA.

According to certain embodiments, the library is a CRISPR library further comprising a nucleic acid sequence encoding an RNA-guided DNA endonuclease enzyme. According to certain exemplary embodiments, the endonuclease is Cas9.

The compositions and methods of the present invention have been exemplified in the model plant Arabidopsis. The large number of gene families in Arabidopsis results in high levels of functional redundancy (O'Malley, R. C. & Ecker, J. R. 2010. Plant J. 61, 928-940). In recent years, genome-scale amiRNA collections have been developed in Arabidopsis and used for forward-genetic screening to identify hidden phenotypes masked by redundant homologous genes (Zhang, Y. et al. 2018. Nat. Commun. 9; Hauser, F. et al. 2013. Plant Cell 25, 2848-2863). However, this strategy generally results in incomplete knockout phenotypes. The CRISPR/Cas9 system is a simple, effective method for generating targeted heritable mutations in the genome and has recently enabled large-scale knockout mutant libraries of single genes to be generated for forward-genetic screens in mammalian (Park, R. J. et al. 2017. Nat. Genet. 49, 193-203; Wang, T., et al. 2014. Science. 343, 80-84) and plant systems (Jacobs, T. B., et al. 2017. Plant Physiol. 174, 2023-2037; Chen, K. et al. 2021. Mol. Plant; Liu, H. J. et al. 2020. Plant Cell 32, 1397-1413; Lu, Y. et al. 2017. Mol. Plant 10, 1242-1245; Meng, X. et al. 2017. Molecular Plant). An important advantage of the CRISPR/Cas9 method is its capacity to simultaneously target multiple genes, whether they are genetically linked or not. The present invention discloses a novel genome-scale approach with the ability to simultaneously target several genes within the same gene family or a functional or molecular pathway. The approach was applied to Arabidopsis. The forward-genetic strategy according to the teachings of the present invention overcomes functional redundancy and enables flexible screening, ranging from a specific functional subgroup to the entire genome. The approach and the library constructed according to the teachings of the invention allows a broad spectrum of functional screens to be readily carried out, thereby significantly impacting current genetic analyses in plants.

Following successful phenotyping and genotyping of Multi-Knock T2 plants, just as in any other genetic approach (e.g., use of alkylating agents, T-DNA, amiRNA), it is critical to validate that the phenotype is indeed driven by the specific mutation. Demonstrating such on-target activity should use the following methods: 1) use of homozygous knockout where Cas9 is crossed out; 2) use of at least two independent mutant lines such as a combination of T-DNA lines or, in cases of genetic linkage, sgRNAs or amiRNA; 3) use of complementation lines to demonstrate phenotype rescue. In agreement, here, we used independent amiRNA and sgRNAs lines to genetically pinpoint the complex and partial redundant activity of PUP7, PUP8 and PUP21.

As exemplified herein, the use of Multi-Knock for gene function discovery in Arabidopsis was validated. The inventors have further shown that the method is applicable in tomato and rice. Thus, the genome-scale multi-targeted mutagenesis system of the present invention can be applied to a variety of plant species. Large-scale Agrobacterium-mediated plant transformations in crops remain a bottleneck due to low transformation efficiency and requirement for labor-intensive tissue culture. Enhancing transformation efficiency, for example, using sgRNA delivery by viral vectors (Ellison, E. E. et al. 2020. Nat. Plants 6, 620-624; Wang, M. et al. 2017. Mol. Plant 10, 1007-1010) or nanoparticle-based carriers (Martin-Ortigosa, S. et al. 2014. Plant Physiol. 164, 537-547; Mitter, N. et al. 2017. Nat. Plants 3), allows the Multi-Knock approach of the present invention to be readily employed in many other plant species.

The term “vector” is used herein as known in the art and refers to a small carrier nucleic acid molecule such as plasmid, virus or other agent that can be manipulated by insertion of a nucleic acid. The term “construct”, as used herein refers to an engineered DNA molecule including one or more nucleotide sequences from different sources. The terms “vector” and “construct” are used herein interchangeably.

The following examples are presented in order to more fully illustrate some embodiments of the invention. They should, in no way be construed, however, as limiting the broad scope of the invention.

### Examples

### Materials and Methods

Plant material and growth conditions. All Arabidopsis plants were derived from the Columbia ecotype and grown in dedicated growth rooms under long-day conditions (16 h light/8 h dark) at 22° C. Arabidopsis Col-0 plants were transformed using Agrobacterium strains (GV3101) by the flower dip method.

Multi-targeted sgRNA design. All 9,350 gene families in the Arabidopsis thaliana genome, encompassing 27,416 genes, were downloaded from the PLAZA 3.0 plant comparative genomics database. Genes belonging to the mitochondrial and chloroplast genomes were filtered out, as well as families with a single family member, leaving 3,892 families of size 2 or more that together encompassed 21,798 genes. The CRISPys software was then applied to each family while accounting for the homologous relationships within each family. Specifically, given a family of genes, a gene tree was reconstructed using a hierarchical clustering algorithm, which clusters the genes according to their sequence similarity. The s^{Ω} design strategy of CRISPys was then recursively applied to each subgroup induced by the gene tree to find the optimal sgRNAs for targeting the desired subfamily. CRISPys was applied using the CFD (Cutting Frequency Determination) score as the scoring function with targeting efficacy threshold of Ω=0.55 and k=12 as the threshold for the number of polymorphic sites. The number of sgRNAs per each subgroup of genes in a given gene tree was limited to 200. The potential sgRNA targets were allowed only for the first two-thirds of the coding sequence. Since CRISPys could assign the same sgRNAs for different subgroups of homologous genes, where one subgroup is a subset of the other one (for example, assuming that {g₁, g₂, g₃} is a subset of homologous genes, and s is an sgRNA that targets this subgroup of genes, the same sgRNA s can also be found for {g₁, g₂}), we considered only one occurrence of the sgRNA.

For each remaining sgRNA, a genome-wide off-target detection was applied. In the context of gene-family cleavage, an off-target is defined as a potential genomic target that is outside the specified gene family, while on-targets are nuclear targets that reside within the family, even though some mismatches may occur between them and the examined sgRNA. To this end, given a specified sgRNA, the Burrows-Wheeler Aligner (BWA) was applied to the Arabidopsis thaliana genome (PLAZA v3) to identify potential nuclear hits. BWA was executed with the command “bwa aln”, with the following parameters: -N, -l 20, -i 0, -n 5, -o 0, -d 3, -k 4, -M 0, -O 1000000, -E 0, thus allowing searching for targets with at most four mismatches and no gaps. Only hits that reside within protein-coding exons were considered off-targets. A potential sgRNA was filtered if it was inferred to cleave an off-target with a CFD score higher than 0.33. We then applied an additional filtering procedure, where we tested the remained sgRNAs for overlapping target regions. A given sgRNA was removed if all its targets overlapped with those of a second potential sgRNA, and the CFD scores of most of these targets were lower. A sgRNA s₁ is defined to overlap with sgRNA s₂ if the positions of all its targets overlap with those of s₂ in at least 10% of the aligned region (i.e., 2 bp).

CRISPR/Cas9 vectors To generate the pRPS5A:Cas9 OLE: CITRIN plasmid, Site-Directed Mutagenesis (NEB-E0554S), was used to eliminate the 3 BsaI sites within the OLE:CITRINE sequence, the following using primers: Fwd-ATGGGCCGAGACAGGGACCAGTACCAGATGTCCGGAC (SEQ ID NO:1) Rev-CATCGGGTACTGGTCCCTGCCGATGATATCGTGATGG (SEQ ID NO:2). The BsaI sites are required for the Golden gate CRISPR library cloning. Next, OLE:CITRINE was cut and ligated from pJET into pRPS5A:Cas9 vector using Mlul and BamHI restriction enzymes. pUBI:Cas9 was generated as described previously. pRPS5A: zCAS9i (Addgene ID: AGM55261) and pEC:Cas9 (Addgene ID: pHEE401) were purchased from Addgene.

Construction of Multi-Knock, multi-targeted CRISPR libraries. The 20-nucleotide sgRNA target sites were appended with the specific adaptors and BsaI sites, as seen in Table 1.

TABLE 1
Example of the sgRNAs Multi-Knock oligonucleotides design

| | Pool | |
|---|---|---|
| Pools | description | Adaptor sequence + sgRNA |
| PKR | Protein | CTGGTCATCATCCTGCCTTTggtctcGattgNNNNNNNNNNNNNNNNNNNN |
| | kinases, | GTTTcGAGACCTGTTATATGGAGGGGGCAAA (82 bp) (SEQ ID NO: 3) |
| | protein | |
| | phosphatases, | |
| | receptors, | |
| | and their | |
| | ligands | |
| BNO | Protein | GGATTATTCATACCGTCCCAggtctcGattgNNNNNNNNNNNNNNNNNNNN |
| | binding small | GTTTcGAGACCAATCAGCCATACCACATTTG (82 bp) (SEQ ID NO: 4) |
| | molecules | |
| CSI | Proteins that | AGAGCTCGTTTAGTGAACCGggtctcGattgNNNNNNNNNNNNNNNNNNNN |
| | form or | GTTTcGAGACCGATGAGTTTGGACAAACCAC (82 bp) (SEQ ID NO: 5) |
| | interact with | |
| | protein | |
| | complexes | |
| | including | |
| | stabilization | |
| | of those | |
| TFB | Transcription | CGTTGGCTACCCGTGATATTggtctcGattgNNNNNNNNNNNNNNNNNNNN |
| | factors | GTTTcGAGACCCTATTCGGCTATGACTGGGC (82 bp) (SEQ ID NO: 6) |
| TEC | Metabolic | TGTAAAACGACGGCCAGTggtctcGattgNNNNNNNNNNNNNNNNNNNN |
| | and other | GTTTcGAGACCCGTAATCATGGTCATAGCTGTT (82 bp) (SEQ ID NO: 7) |
| | enzymes | |
| | catalyzing | |
| | transfer | |
| | reactions | |
| PEC | Active | GGAAACAGCTATGACCATGggtctcGattgNNNNNNNNNNNNNNNNNNNN |
| | catalytic | GTTTcGAGACCGTCGTGACTGGGAAAACC (79 bp) (SEQ ID NO: 8) |
| | proteins, | |
| | mainly | |
| | enzymes | |
| UNC | Genes for | CGACTCACTATAGGGAGAGCGGCggtctcGattgNNNNNNNNNNNNNNNNNNNN |
| | which the | GTTTcGAGACCCTGCCATGGAAAATCGATGTTCTT (89 bp) (SEQ ID NO: 9) |
| | function is | |
| | not known or | |
| | cannot be | |
| | inferred | |
| DMF | A protein | TCCTCCGCTTATTGATATGCggtctcGattgNNNNNNNNNNNNNNNNNNNN |
| | with diverse | GTTTcGAGACCCCTTGTTACGACTTTTACTTCC (84 bp) (SEQ ID NO: 10) |
| | functional | |
| | annotation | |
| | not found in | |
| | the other | |
| | categories | |
| HEC | Hydrolytic | GCGAGAGTAGGGAACTGCggtctcGattgNNNNNNNNNNNNNNNNNNNN |
| | enzymes, | GTTTcGAGACCGTGTCTCAAAATCTCTGATGTT (82 bp) (SEQ ID NO: 11) |
| | excluding | |
| | protein | |
| | phosphatases | |
| TRP | Transporters | CCAAGCTATTTAGGTGACACggtctcGattgNNNNNNNNNNNNNNNNNNNN |
| | | GTTTcGAGACCTAGAAGGCACAGTCGAGG (80 bp) (SEQ ID NO: 12) |

* N = A, G, T or C
** Marked in bold are adaptor sequences; Marked in Italic are sgRNA molecules, wherein each sgRNA is to comprises a unique sequence; ggtctcGattg (SEQ ID NO: 60)/GTTTcGAGACC (SEQ ID NO: 61)-BsaI sites.

Synthesis of the 59,129 DNA oligonucleotides corresponding to the sgRNAs was performed by Twist Bioscience, and the oligonucleotide library was concentrated to 500 ng. The single-stranded oligonucleotide pool was converted to double-stranded DNA by PCR using the high-fidelity Phusion polymerase (NEB) using 12 to 15 cycles of PCR to avoid proofreading mistakes. PCR was conducted using the following conditions: 98° C. for 30 s; 15 cycles of 98° C. for 30 s, 60° C. for 30 s, and 72° C. for 15 s; and a final extension at 72° C. for 10 min. For each family pool, about 6 tubes of 50 μl-volume amplification reactions with a total of 15 ng single-stranded oligonucleotide pool as a template and the specific primers for adaptors (Table 2) were used, and the PCR products were purified with a NucleoSpin Gel and PCR clean up Kit (Macherey-Nagel).

TABLE 2
Primers for amplification of subgroups
of sgRNA library.

| | Forward primer | Reverse primer |
|---|---|---|
| Pools | (5′-3′) | (5′-3′) |
| PKR | CTGGTCATCATCCTGCCTTT | TTTGCCCCCTCCATATAACA |
| | (SEQ ID NO: 13) | (SEQ ID NO: 14) |
| BNO | GGATTATTCATACCGTCCCA | CAAATGTGGTATGGCTGATT |
| | (SEQ ID NO: 15) | (SEQ ID NO: 16) |
| CSI | AGAGCTCGTTTAGTGAACCG | GTGGTTTGTCCAAACTCATC |
| | (SEQ ID NO: 17) | (SEQ ID NO: 18) |
| TFB | CGTTGGCTACCCGTGATATT | GCCCAGTCATAGCCGAATAG |
| | (SEQ ID NO: 19) | (SEQ ID NO: 20) |
| TEC | TGTAAAACGACGGCCAGT | AACAGCTATGACCATGATT |
| | (SEQ ID NO: 21) | ACG |
| | | (SEQ ID NO: 22) |
| PEC | GGAAACAGCTATGACCATG | GGTTTTCCCAGTCACGAC |
| | (SEQ ID NO: 23) | (SEQ ID NO: 24) |
| UNC | CGACTCACTATAGGGAGAG | AAGAACATCGATTTTCCAT |
| | CGGC | GGCAG |
| | (SEQ ID NO: 25) | (SEQ ID NO: 26) |
| DMF | TCCTCCGCTTATTGATATGC | GGAAGTAAAAGTCGTAAC |
| | (SEQ ID NO: 27) | AAGG |
| | | (SEQ ID NO: 28) |
| HEC | GCGAGAGTAGGGAACTGC | AACATCAGAGATTTTGAG |
| | (SEQ ID NO: 29) | ACAC |
| | | (SEQ ID NO: 30) |
| TRP | CCAAGCTATTTAGGTGACA | CCTCGACTGTGCCTTCT |
| | CGG | AGG |
| | (SEQ ID NO: 31) | (SEQ ID NO: 32) |

The purified DNA products were digested with BsaI restriction enzyme and ligated into the desired Cas9 expression constructs using the Golden Gate cloning method. Golden Gate assembly was performed as follows: 35 cycles of 37° C. for 5 min and 16° C. for 5 min; 50° C. for 20 min; and 80° C. for 20 min. Four 20-μl ligation reactions were combined, and 20 bacterial transformations were carried out using 4 μl of ligation reaction and 50 μl Top10 chemically competent E. coli per transformation according to the manufacturer's instructions. The 20 transformations were combined and plated onto seven LB agar plates (145×20 mm, Greiner Bio-one) supplemented with the relevant antibiotics. Colonies were validated using colony PCR and Sanger sequencing individually, then bacteria from all plates were scraped off and combined. The plasmid DNA was purified with a Plasmid Maxi kit (Qiagen) to produce the CRISPR libraries. In order to verify these plasmid pools, PCR products amplified with the primers listed in Table 3 from the CRISPR libraries were sequenced on an Illumina NovaSeq 6000 with the PE150 mode.

TABLE 3
Primers for NGS PCR amplification and
sgRNAs genotyping in transgenic plants.

| Cas9 | Forward | Reverse |
|---|---|---|
| expression | primer | primer |
| constructs | (5′-3′) | (5′-3′) |
| pRPS5A: | TACTAGATCG | CCGACTCGGT |
| zCas9i | ACGCTACTAG | GCCACTTTT |
| | (SEQ ID NO: 33) | (SEQ ID NO: 34) |
| pUBI: | CCCCTGGGAAT | CCGACTCGGT |
| Cas9 | CTGAAAGAAG | GCCACTTTT |
| | (SEQ ID NO: 35) | (SEQ ID NO: 36) |
| PEC: | CCCCTGGGAAT | CCGACTCGGT |
| Cas9 | CTGAAAGAAG | GCCACTTTT |
| | (SEQ ID NO: 37) | (SEQ ID NO: 38) |
| pRPS5A: | CCCCTGGGAAT | CCGACTCGGTGC |
| Cas9 with | CTGAAAGAAG | CACTTTT |
| OLE: | (SEQ ID NO: 39) | (SEQ ID NO: 40) |
| CITRIN | | |

The number of reads per sgRNA sequence was quantified from the raw sequencing data using the Biopython package in the Python programing language.

Generation of four transportome CRISPR libraries. The four transportome CRISPR plasmids were transformed into Agrobacterium tumefaciens strain GV3101 using electroporation. In brief, for each library, around 20 tubes of GV3101 competent cells (80 μl) were incubated on ice with ˜1 μg plasmid in each tube for 5 min and electroporated using a MicroPulser (Bio-Rad Laboratories; 2.2 kV, 5.9 ms). Immediately after electroporation, 700 μl LB medium was added, and samples were shaken for 1.5-2 h at 28° C. Agrobacterium was then plated on LB agar plates (145×20 mm, Greiner Bio-one) containing the relevant antibiotics for 2 days at 28° C. in the dark. Each Agrobacterium transportome CRISPR library was transformed into six trays of Arabidopsis Col-0 plants. T1 Seeds were collected in bulk. After transformant plant selection, transgenic plants for each transportome CRISPR library were propagated, and T2 seeds were collected. We collocated 2,000 independent T2 lines of pRPS5A:zCas9i individually. pUBI:Cas9, pEC:Cas9, pRPS5A:Cas9 OLE: CITRIN lines were collected in bulks of 10 plants. Phenotypic screens were carried out on the T1 and T2 generations.

Arabidopsis transformation and heat-shock treatment. The Agrobacterium colonies from all plates were scraped off and added into 1 L LB medium with 25 μg/ml gentamycin, 25 μg/ml rifampicin, and vector-specific antibiotic, followed by incubation at 28° C. for 16-24 hours. Agrobacterium was harvested by centrifugation for 10 min at 5,500 rpm, the supernatant was discarded, and the bacteria pellet was resuspended in ˜400 ml inoculation medium containing 0.5×MS (Duchefa Biochemie), 5.0% sucrose, and 0.05% Tween-20 (Sigma-Aldrich). Arabidopsis flowers were then sprayed with the bacterial solution. After spraying, plants were kept in the dark overnight and grown until siliques ripened and dried. T1 seeds were collected in bulk. The T1 seeds of the pEC:zCas9 library were sown on MS media containing hygromycin (25 μg/ml) for the transformant plant selection, whereas the T1 seeds of the other three transportome CRISPR libraries were sown on soil and sprayed with BASTA for selection at the age of 2 weeks. Except of T1 plants of pRPS5A:Cas9 OLE:CITRINE, all T1 transgenic plants were subjected to repeated heat stress treatments as previously described with slight modifications. The plants that were subjected to heat stress were treated as follows: After resistance selection and 4 days of acclimation to the soil, the seedlings were transferred to growth chambers at 32° C. for 24 h, followed by a 48 h recovery at 22° C. (3-day period). This heat stress cycle was performed four times during the vegetative phase of growth. The plants were then grown at 22° C. from that point on.

CRISPR/CAS9 and amiRNA cloning. The 20 nt protospacer (CTCTACTTTCTCCCTCATCT, SEQ ID NO:58) was picked to target PUP7 (AT4G18197), PUP8 (AT4G18195) and PUP21 (AT4G18205) at once. The oligos (FW: attgCTCTACTTTCTCCCTCATCT (SEQ ID NO:41); REV: aaacAGATGAGGGAGAAAGTAGAG (SEQ ID NO:42) were annealed and cloned into the pRPS5A: zCAS9i (Addgene: AGM55261) using the Golden Gate cloning method. In brief, the oligos were incubated at 95° C. for 5 mins and cooled at RT for 20 mins. The annealed oligos and the pRPS5A: zCAS9i were added in the following reaction (20 μl): 3 μl of annealed oligos; ˜ 150 ng of CAS9 vector; 1 μl T4 ligase (400,000 units/ml, NEB); 1 μl BsaI-HF v2 (20,000 units/ml, NEB); Cutsmart buffer (NEB) and T4 ligase buffer (NEB). Golden Gate assembly was performed as follows: 35 cycles of 37° C. for 5 min and 16° C. for 5 min; 50° C. for 20 min; and 80° C. for 20 min. 1/10 of the reaction was transformed into E. coli DH5a.

To generate the 35S: amiRNA-PUP7/8/21 vector, the amiRNA319 backbone sequence with miR targeting PUP7, PUP8 and PUP21 (MiR-sense: TATCATGGAAAACTGTCACTG, SEQ ID NO:59) was synthesized by Syntezza Bioscience Ltd. and cloned into the pH2GW7 destination vector using the Gateway system.

Genotyping. To identify the sgRNA of transgenic plants, genomic DNA from young leaf tissue was extracted by grinding 1-2 leaves into 400 μl Extraction Buffer (200 mM Tris-HCl, pH 8.0, 250 mM NaCl, 25 mM EDTA, and 0.5% SDS). After 1-min centrifugation at 13,000 rpm, 300 μl supernatant was transferred to a new Eppendorf tube and mixed with 300 μl isopropanol, followed by centrifugation for 10 min at maximum speed. The supernatant was removed and the DNA pellets were washed with 70% ethanol and then resuspended in 50 μl of water. The PCR amplified using the primers listed in Table 3 was identified using Sanger sequencing.

T-DNA lines for the single mutants, listed in Table 4, were ordered from Gabi Kat (https://www.gabi-kat.de) and The Arabidopsis Information Resource (https://www.arabidopsis.org/). Primers for the T-DNA genotyping were designed using the T-DNA Primer Design Tool powered by Genome Express Browser Server (http://signal.salk.edu/tdnaprimers.2.html). Homozygous mutants were selected by PCR performed with primers listed in Table 4.

TABLE 4
Genotyping primers for T-DNA lines

| | T-DNA | Primer | Primer sequence |
|---|---|---|---|
| Gene | Line | name | (5′-3′) |
| PUP7 | SALK_ | LP | TAAAGACACCCTCCATGTTCC |
| | 084103 | | (SEQ ID NO: 43) |
| | | RP | AAATTGACAATGAACGTTGCC |
| | | | (SEQ ID NO: 44) |
| | | LB | TAGCATCTGAATTTCATAACC |
| | | | AATCTCGATACAC |
| | | | (SEQ ID NO: 45) |
| PUP8 | SALK_ | LP | TCGTTGTTGACTTCCAAATCC |
| | 137526 | | (SEQ ID NO: 46) |
| | | RP | TGAGGATCAGTTGTACCAGGG |
| | | | (SEQ ID NO: 47) |
| | | LB | TAGCATCTGAATTTCATAACC |
| | | | AATCTCGATACAC |
| | | | (SEQ ID NO: 48) |
| PUP21 | GK- | LP | AGCAAAAGGTACCTGATGACG |
| | 288E11 | | (SEQ ID NO: 49) |
| | | RP | CTTTTCGTCGAGGTAGTGCTG |
| | | | (SEQ ID NO: 50) |
| | | LB | ATAATAACGCTGCGGACATCT |
| | | | ACATT |
| | | | (SEQ ID NO: 51) |

35S: YFP-PUPs cloning. PUP7 genomic DNA, PUP8-CDS and PUP21-CDS were amplified with Phusion High-fidelity Polymerase (NEB) using the primers list in Table 5.

TABLE 5
PUP cloning

| Cloning | | |
|---|---|---|
| primers/ | Forward primer | Reverse prime |
| Gene | (5′-3′) | (5′-3′) |
| PUP7 | caccATGGACAGAT | TCACACACTTTGT |
| genomic | CTCAAGAACACTAT | ATGTTTGTGTGA |
| | GCC | CC |
| | (SEQ ID NO: 52) | (SEQ ID NO: 53) |
| PUP8 | caccATGGAAATAAC | TCATACACTATGT |
| CDS | TCAAGTAATCTATGT | ATGTTTGTGTGA |
| | | CCTTCC |
| | (SEQ ID NO: 54) | (SEQ ID NO: 55) |
| PUP21 | caccATGGGCATATCT | TCATAAAGTTTG |
| CDS | CAAGTACACTATTGC | TGTTTCTTCCTCA |
| | (SEQ ID NO: 56) | ACAGGTT |
| | | (SEQ ID NO: 57) |

PUP7 genomic sequence with intron, PUP8, and PUP21 coding regions was cloned into pENTER/D-TOPO (Invitrogen K2400), verified by sequencing, and subsequently cloned into the binary destination vector (pH7WGY2) using LR Gateway reaction (Invitrogen 11791). p35S: YFP-PUP7, p35S: YFP-PUP8, and p35S: YFP-PUP21 were generated using the pH7WGY2 vector and were selected using spectinomycin in Escherichia coli and hygromycin in plants.

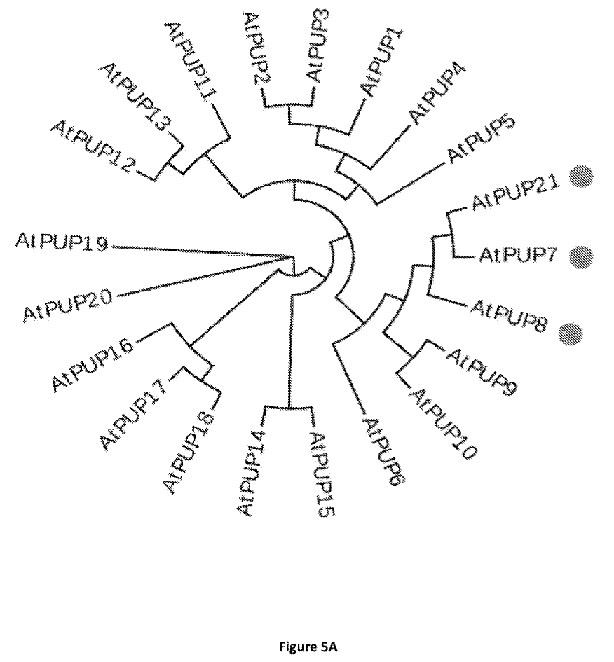

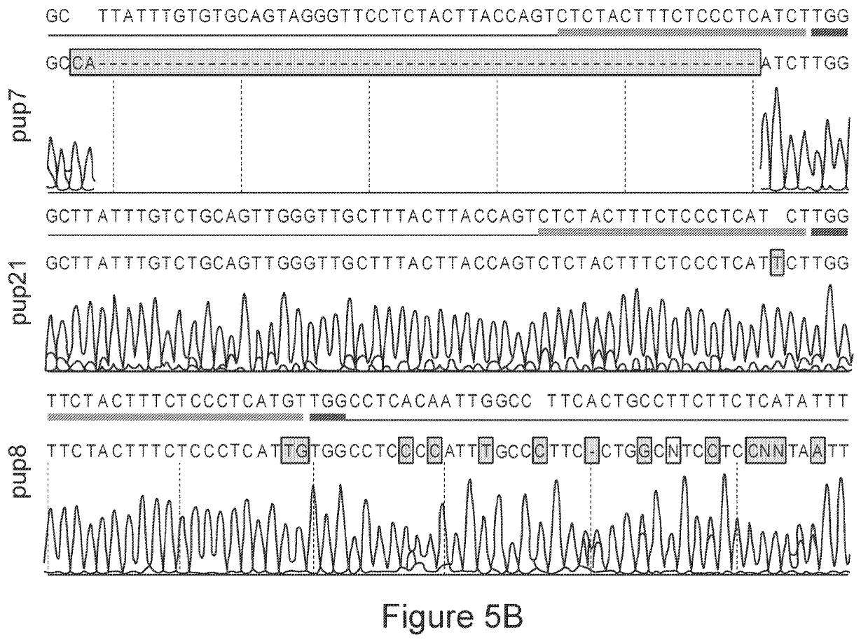

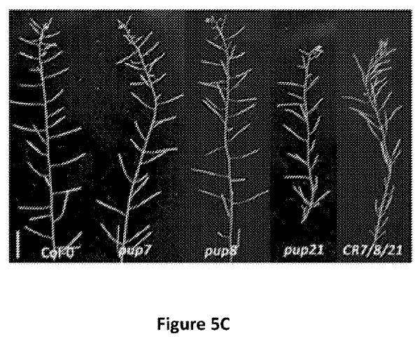

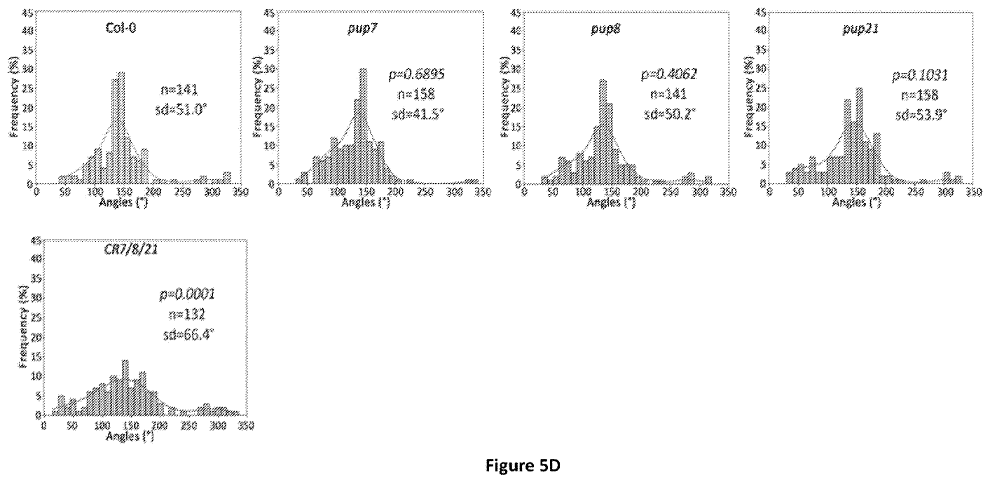

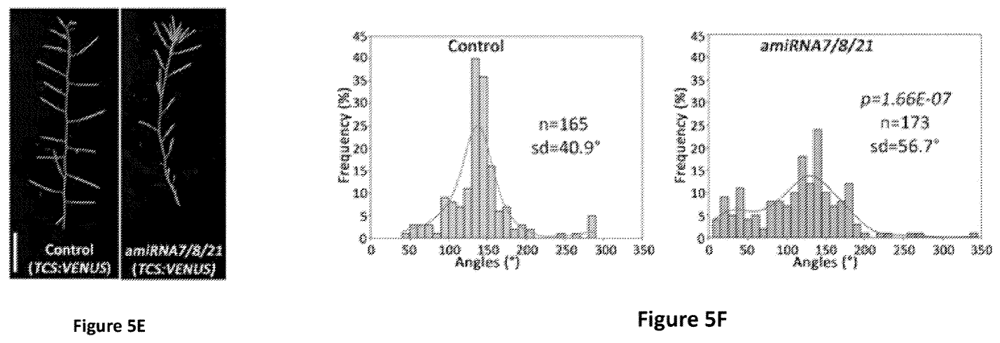

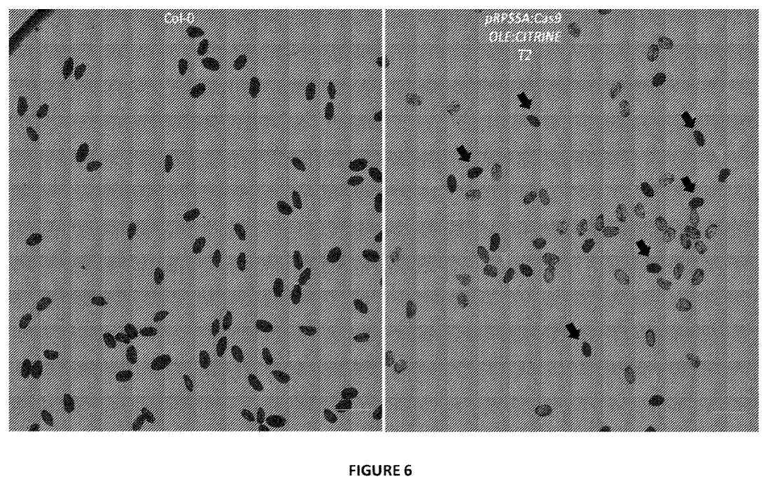

Phylogenetic tree. A phylogenetic tree of Arabidopsis PUP family members, based on protein sequences, was constructed using Phylogeny.fr (http://www.phylogeny.fr/) with “one-click” mode. The previously unreported PUP9 protein (AT4G18220), a close paralog of PUP10, was identified and added to the phylogenetic analysis (FIG. 5A).

Measurements of silique divergence angles. Angles separating successive siliques on the main inflorescence stem were quantified using a protractor as previously described. The divergence angle was measured between the insertion points of two successive floral pedicels. Phyllotaxy orientation can be either clockwise or anticlockwise.

### Example 1: Design of the Multi-Knock Multi-Targeted, CRISPR-Based, Genome-Scale Genetic Toolbox

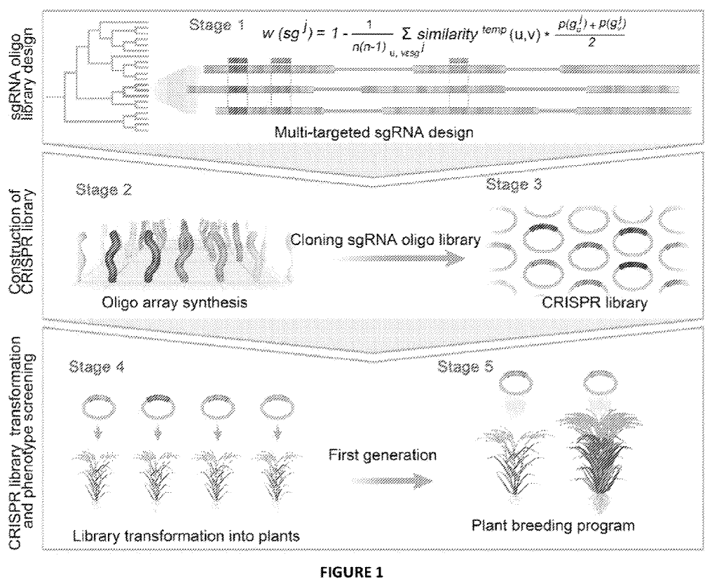

The high similarity in coding sequences within plant gene families often results in complete or conditional functional redundancy, leading to substantial phenotypic buffering. In order to overcome functional redundancy, we developed Multi-Knock, a new toolbox to knock out gene families at a genome-scale using a CRISPR/Cas9-based strategy (FIG. 1).

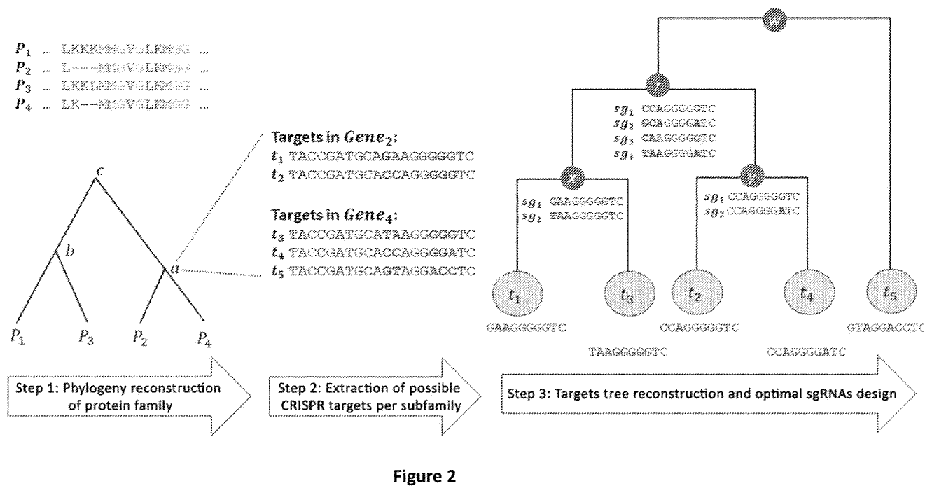

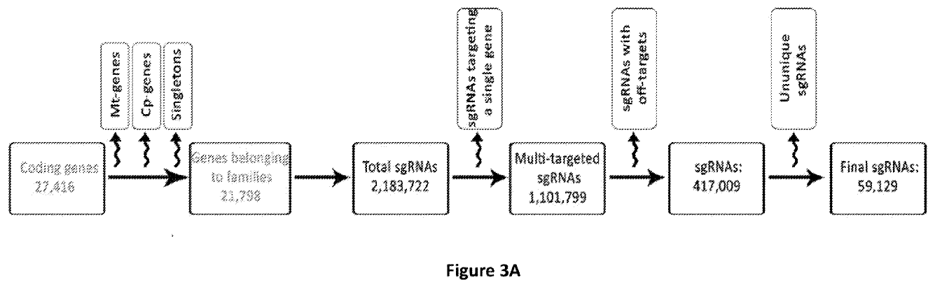

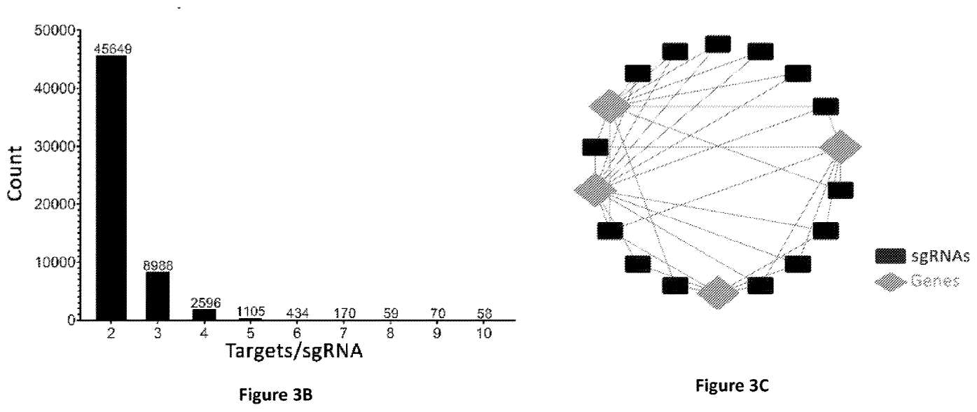

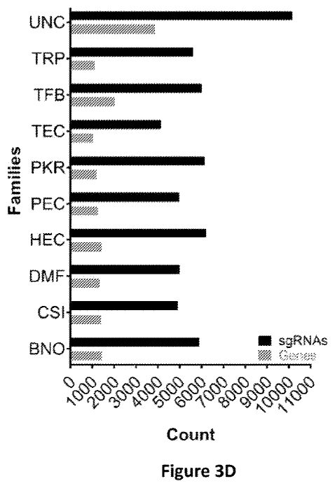

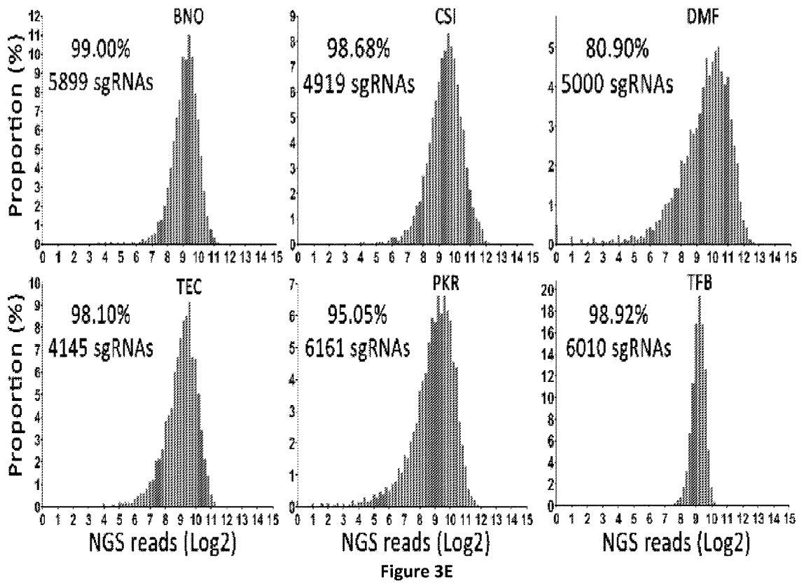

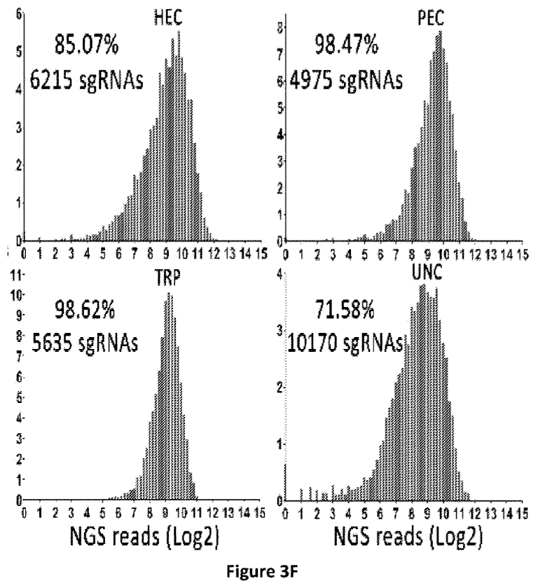

To construct a genome-scale library of sgRNAs that would potentially target multiple members from the same family, all gene families in Arabidopsis thaliana genome (TAIR10), encompassing 27,416 protein-coding genes, were downloaded from the PLAZA 3.0 plant comparative genomics database. Following the filtration of mitochondrial and chloroplast genes, as well as singletons (i.e., genes without any family members), 21,798 genes remained, belonging to 3,892 families of size 2 or more. We then designed a set of sgRNAs that would optimally target multiple members of each gene family while accounting for the similarity among family members (FIG. 2). Specifically, a phylogenetic reconstruction strategy was used to hierarchically organize each family into a tree structure, such that a homologous subgroup of genes that are more closely related are placed closer to each other on the tree. The optimal sgRNAs that could most efficiently target multiple members of each subgroup were designed using the CRISPys algorithm. Since CRISPys could potentially design the same sgRNAs for different subgroups of the same family, we considered only one occurrence of each sgRNA (FIG. 2). This procedure resulted in a total of 2,183,722 sgRNAs. Next, we removed sgRNAs that targeted only a single gene with high efficiency, resulting in 1,101,799 sgRNAs. We then removed sgRNAs with potential high off-target activity towards unintended Arabidopsis coding regions and filtered sgRNAs with overlapping targets. This resulted in a total of 59,129 sgRNAs targeting 16,152 genes (˜74% of all protein-coding genes that belong to families) (FIG. 3A). Of the 59,129 sgRNAs, 98.7% target two to five genes; the rest target six to ten genes (FIG. 3B). This set of sgRNAs creates a robust library where every sgRNA targets multiple genes, and every gene is targeted by multiple sgRNAs (FIG. 3C).

### Example 2: Construction of Multi-Targeted CRISPR Sub-Libraries for Specific Functional Groups

In order to increase the flexibility of the Multi-Knock library and enable targeted forward-genetics screens, the 59,129 sgRNAs were classified into 10 groups according to the protein functions of their putative target genes, thus creating the following ten sub-libraries: transporters (TRP: 1,123 genes and 5,635 sgRNAs); protein kinases, protein phosphatases, receptors, and their ligands (PKR: 1,190 genes and 6,161 sgRNAs); transcription factors and other RNA and DNA binding proteins (TFB: 2,042 genes and 6,010 sgRNAs); proteins binding small molecules (BNO: 1,443 genes and 5,899 sgRNAs); proteins that form or interact with protein complexes including stabilizing factors (CSI: 1,399 genes and 4,919 sgRNAs); hydrolytic enzymes (enzyme classification [EC] class 3), excluding protein phosphatases (HEC: 1,438 genes and 6,215 sgRNAs); metabolic enzymes and enzymes (EC class2) that catalyze transfer reactions (TEC: 1,041 genes and 4,145 sgRNAs); catalytically active proteins, mainly enzymes (PEC: 1,252 genes and 4,975 sgRNAs); proteins with diverse functional annotations not found in the other categories (DMF: 1,343 genes and 5,000 sgRNAs); and proteins of unknown function or cannot be inferred (UNC: 3,881 genes and 10,170 sgRNAs) (FIG. 3D, Table 7).

In order to facilitate the creation of the sub-libraries, adaptors of 38 to 47 nucleotides in length were added that were unique to each sub-library (Table 2). We amplified each sub-library using primers complementary to the specific adaptors and used the Golden Gate method to clone the sgRNA sub-libraries into the intronized zCas9 vector (pRPS5A:zCas9i). The intronized Cas9 has a number of introns integrated into the maize codon-optimized Cas9; these introns have a significant positive effect on Cas9 genome editing efficiency in Arabidopsis.

More than 2.0×10⁵ clones of each sub-library growing on the selection plates were harvested, and plasmid DNA from each sub-library was isolated. In order to evaluate library quality, each library was deep sequenced in a 150 paired-end mode (PE150). The sequencing data showed that more than 95% of the designed sgRNAs in our libraries were present, with the exception of sgRNAs in three sub-libraries (DMF, HEC, and UNC) that exhibited lower coverage percentages (80.90%, 85.07%, and 71.58% coverage, respectively) (FIGS. 3E-3F). Importantly, the sgRNAs frequencies in the sub-libraries showed a narrow bell-shaped distribution (FIGS. 3E-3F), indicating that no individual sgRNA were overly enriched. All libraries will be available to the community as an open-access resource. Together, these quality control analyses indicate that the Multi-Knock CRISPR sub-libraries are ready to be used in plants for functional analysis.

TABLE 7
Overview of sgRNAs and gene numbers per family

| | Targeted genes per family | Genes per | sgRNAs |
|---|---|---|---|
| Families | (Percent Coverage (%)) | family | per family |
| BNO | 1443 (76.1%) | 1896 | 5899 |
| CSI | 1399 (82.7%) | 1692 | 4919 |
| DMF | 1343 (71.0%) | 1891 | 5000 |
| HEC | 1438 (84.3%) | 1706 | 6215 |
| PEC | 1252 (82.6%) | 1515 | 4975 |
| PKR | 1190 (84.7%) | 1405 | 6161 |
| TEC | 1041 (86.0%) | 1211 | 4145 |
| TFB | 2042 (78.2%) | 2611 | 6010 |
| TRP | 1123 (84.6%) | 1327 | 5635 |
| UNC | 3881 (59.3%) | 6544 | 10170 |

### Example 3: Multi-Targeted Transportome Analysis

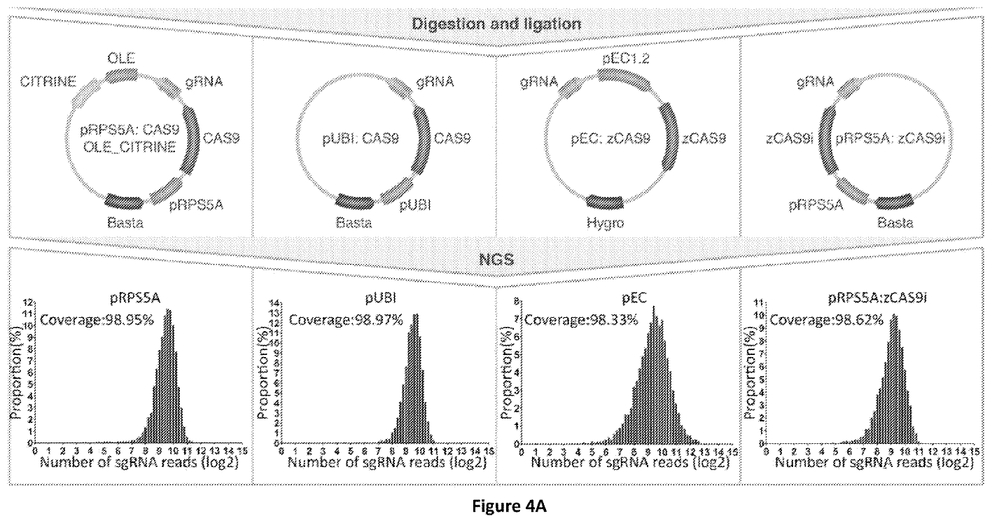

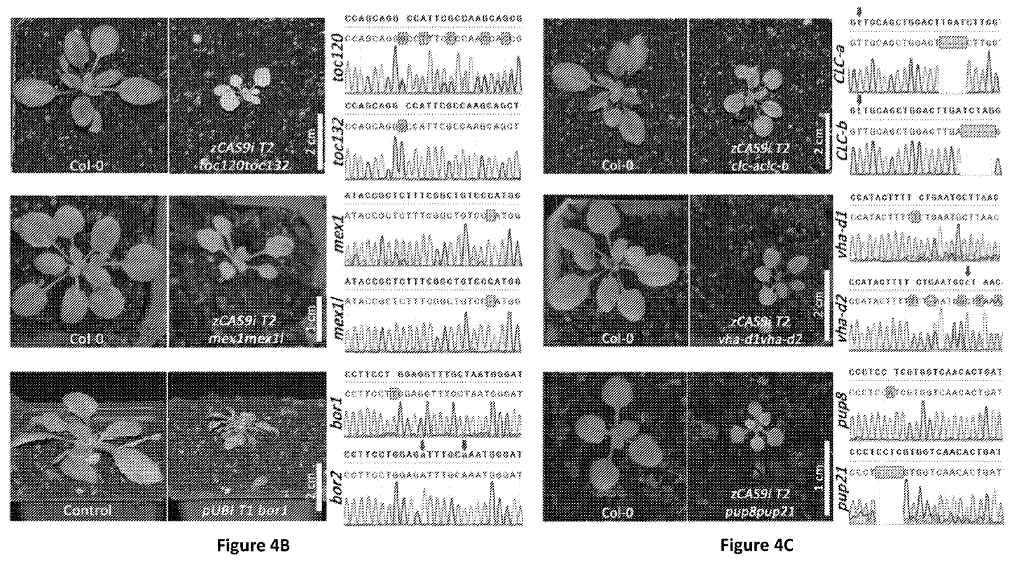

In order to demonstrate that the Multi-Knock approach overcomes redundancy in forward-genetics screens in planta, we chose to focus on the plant transportome using the TRP sub-library. Transporter families in plants are generally large and relatively uncharacterized genetically. To expand the functional utility of our tool, we cloned the 5,635 sgRNA sequences into four different Cas9 vectors to create independent TRP-sub-libraries, varying in their Cas9 type, the promoter driving the Cas9, and resistance in plants: pRPS5A:zCas9i library describes above, which results in high Cas9 genome-editing activity in Arabidopsis (Grützner, R. et al. 2021. Plant Commun. 2, 1-15); pRPS5A:Cas9 with OLE: CITRIN carries BASTA resistance and allows selection of Cas9 in seeds using a fluorescent Citrine protein (Tsutsui, H. & Higashiyama, T. Pkama-Itachi 2017. Plant Cell Physiol. 58, 46-56); the commonly used pUBI:Cas9 also imparts BASTA resistance and pEC:Cas9 carries kana resistance and allows mutation specifically in the egg cells to avoid somatic mutations. The four sub-libraries were cloned and deep-sequenced to evaluate sgRNA coverage and frequency. Coverage was higher than 98%, with a Gaussian distribution for all four libraries (FIG. 4A).

The four TRP-sub-libraries were transformed into Arabidopsis Col-0 plants yielding about 3,500 transgenic T1 plants (pUBI:Cas9, 500 lines; pEC:Cas9, 500 lines; pRPS5A:Cas9 OLE: CITRIN, 500 lines; and pRPS5A:zCas9i 2,000 lines). To increase on-target mutagenesis in plants, pUBI:Cas9, pEC:Cas9, and pRPS5A:zCas9i T1 plants were subjected to repeated mild heat stress as previously described with slight modifications. 2,000 T1 lines were collected individually for the pRPS5A:zCas9i library. pUBI:Cas9, PEC: Cas9, and pRPS5A:Cas9 OLE: CITRIN libraries were each collected in bulks of 10 plants. T1 lines showing dramatic phenotypes were marked, and phenotypes reproducibility was verified. Multiple lines had reproducible defects in leaf color, rosette size, plant height, and flowering time. Importantly, the screen recovered previously reported phenotypes of mutants affected in transporters. For example, we isolated a plant with pale, bleached, and small size shoot. Extracting DNA, amplifying the sgRNA cassette and sequencing, revealed that it putatively targets TOC132 and TOC120 (Translocon Outer Complex proteins) (FIG. 4B). Sanger sequencing of TOC132 and TOC120 revealed that frameshift mutations occurred at the sgRNA target sites in these two genes (FIG. 4B). The phenotype we observed indeed mimicked the toc132, toc120 double mutant phenotype that was previously characterized. In addition, the phenotypes of sgRNA targeting two maltose transporters (MEX1 and MEX1-Like) were in agreement with that of mex1 mutant as described previously (FIG. 4B). In this case, the phenotype of the double mutant was not dramatically enhanced compared to the single MEX1. However, T1 plants targeting genes encoding two boron transporters (BOR1 and BOR2) were identified as double bor1, bor2 knockouts, and had growth inhibition phenotypes (FIG. 4B), likely enhancing the bor1-1 mutant-plants. However, most of the phenotypes we observed were driven by previously undescribed genes. For example, plants expressing a single sgRNA resulted in deletions in clc-a, clc-b (Chloride Channels), or vha-d1, vha-d2 (Vacuolar-type H+-ATPases) or pup8, pup21 (Purin Permeases), all showing smaller rosette size than Col-0 plants (FIG. 4C). At this stage, we do not know whether the phenotypes are a result of an on-target activity, and further genetic validation is needed to rule out off-target effects. Such genetic validation was carried out below for the PUP candidates. Notably, the Multi-Knock seed collection we generated here will be available to the community as an open-access resource for any type of forward-genetic screen. Together, the results demonstrate the strength of the Multi-Knock strategy in exposing novel phenotypic plasticity.

### Example 4: Multi-Knock Screen Revealed Partially Redundant Tonoplast-Localized PUP Cytokinin Transporters

As noted above, the Multi-Knock transportome-scale screen identified a shoot growth inhibition phenotype caused by PUP8 and PUP21 loss-of-function (FIG. 4C). The two unstudied proteins are members of the purine permease (PUP) family, which consists of 21 genes (FIG. 5A). Most of the genes in the PUP Arabidopsis family have not been characterized, but PUP14 reportedly encodes for a plasma membrane cytokinin transporters. In addition to plasma membrane-localized PUP14, PUP1 and PUP2 were also identified as cytokinin transporters in Arabidopsis. In rice, OsPUP1 and OsPUP7 were shown to localize on the endoplasmic reticulum (ER), while OsPUP4 was localized to the plasma membrane. Cytokinins are plant hormones essential for meristem maintenance and additional physiological and developmental processes, such as cell division, lateral root formation, leaf senescence, embryo development and adaptive responses to heat and drought stresses. Because cytokinin biosynthesis, catalyzed by isopentenyl-transferases, does not occur throughout the plant but is limited to certain tissues only, cytokinins are translocated through the plant by diffusion and/or through active transport mechanisms. There is a complete genetic linkage between the PUP7, PUP21, and PUP8 genes, and phylogenetic analysis of PUPs in Arabidopsis showed that these three genes form a monophyletic clade (FIG. 5A). Similar to PUP8 and PUP21, the function of PUP7 is unknown.

To characterize the activity of PUP7, PUP21, and PUP8, we isolated single PUP7, PUP21, and PUP8 T-DNA homozygous lines. The single pup7 (SALK_084103) and pup8 (SALK_137526) mutants showed no morphological differences compared to Col-0. pup21 (GABI_288E11) mutant also did not show a phenotype in the vegetative stage compared to Col-0, and presented only a mild plant height phenotype after bolting (data not shown). To validate the potentially redundant on-target activity PUP7, PUP21, and PUP8 as revealed by the PUP8 and PUP21 loss-of-function line (FIG. 4C), we cloned a multiplexed CRISPR construct targeting PUP7, PUP21, and PUP8 (CRISPR7/8/21). CRISPR7/8/21 showed frameshift mutations in PUP7, PUP21, and PUP8 (FIG. 5B) and exhibited a small rosette size and a perturbed phyllotaxis phenotype with a strong increase in the occurrence of abnormal angles between consecutive organs (FIG. 5C, 5D). Cytokinin response was shown to regulate the spatial distribution of lateral organs along the stem or phyllotaxis. To further validate the on-target activity of PUP7, PUP21, and PUP8 we generated a PUP7, PUP21, and PUP8 multi-targeted amiRNA line (amiRNA7/8/21). amiRNA7/8/21 showed reduced expression of PUP7, PUP21, and PUP8 (data not shown). In agreement with the CRISPR7/8/21 triple mutant, the amiRNA7/8/21 line exhibited a small rosette size and a significantly perturbed phyllotaxis (FIG. 5E, 5F). This result suggests that PUP7, PUP21, and PUP8 redundantly regulate shoot growth and phyllotaxis.

### Example 5: Multi-Knock, Multi-Targeted, CRISPR-Based, in Tomato

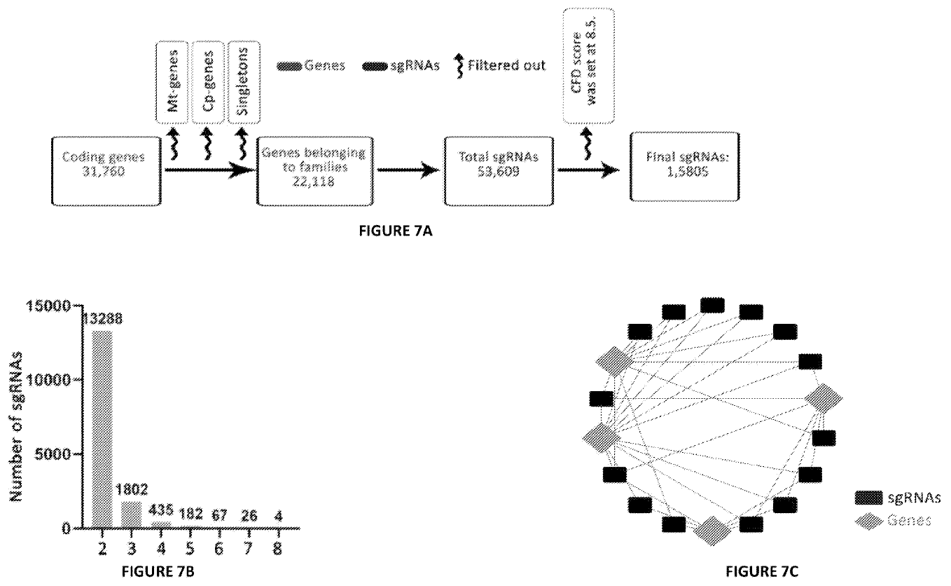

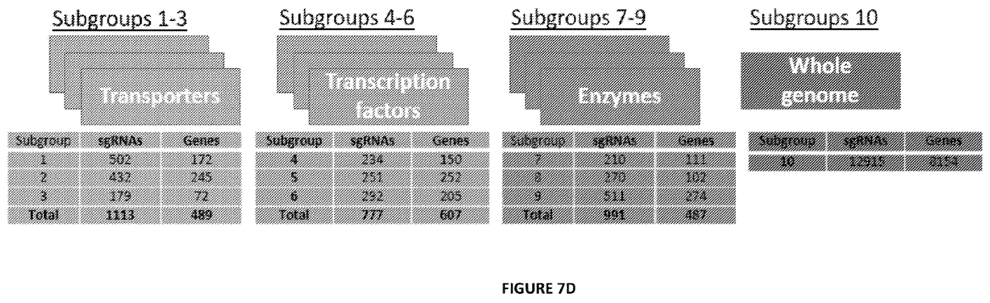

Computational design of a standard library: one sgRNA per construct—The first library for use in tomato was designed and synthesized. The obtained library includes 15,804 sgRNAs targeting 2-8 genes from the same family, and sgRNAs likely to have off-target effects were removed during the design process. In total, 13,590 genes were included in the library (FIG. 7), such that each sgRNA targets multiple genes and nearly all genes are targeted by multiple sgRNAs. The library was then divided into 10 sub-libraries, each directed towards a different functional class of proteins. Our experimental analyses, detailed below, were focused in planta on the transportome sub-library targeting transporter genes to reveal phenotypes related to nutrient uptake.

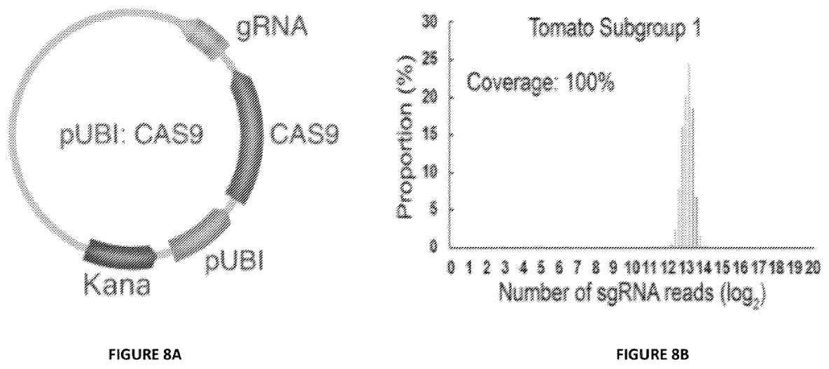

Library synthesis and cloning, including 15,000 sgRNA constructs—To confirm complete coverage and equal representation of sgRNAs, we have deep-sequenced all 10 tomato sub-libraries. The data showed 100% coverage and bell-shaped distribution of equal sgRNA representation in the library (FIG. 8).

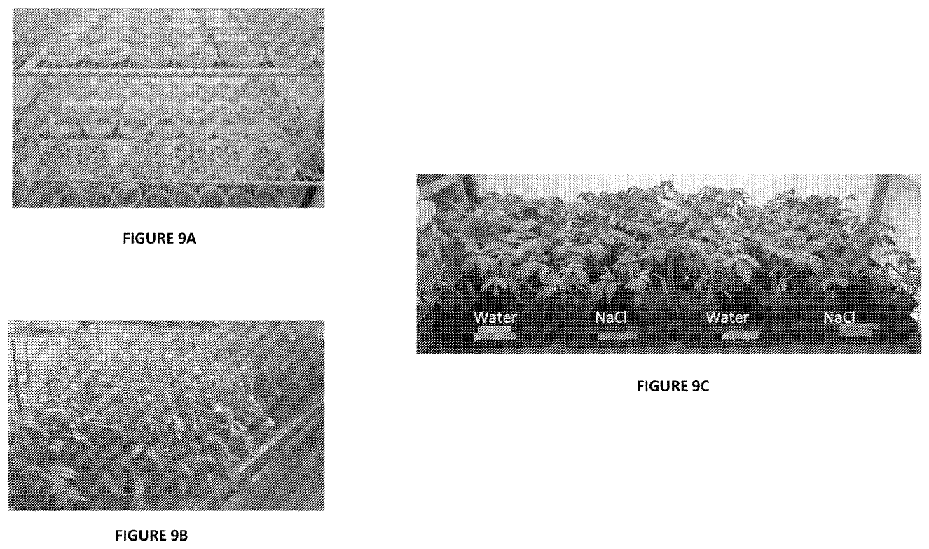

Transformation—The tomato plants were transformed with the transportome multi-targeted CRISPR sub-library 1, which contains 400 sgRNAs. We chose to work with tomato M82 (sp-, determinate tomato mutated in SELF-PRUNING 25 cultivar). We generated over 150 independent tomato lines using tissue culture (FIG. 9).

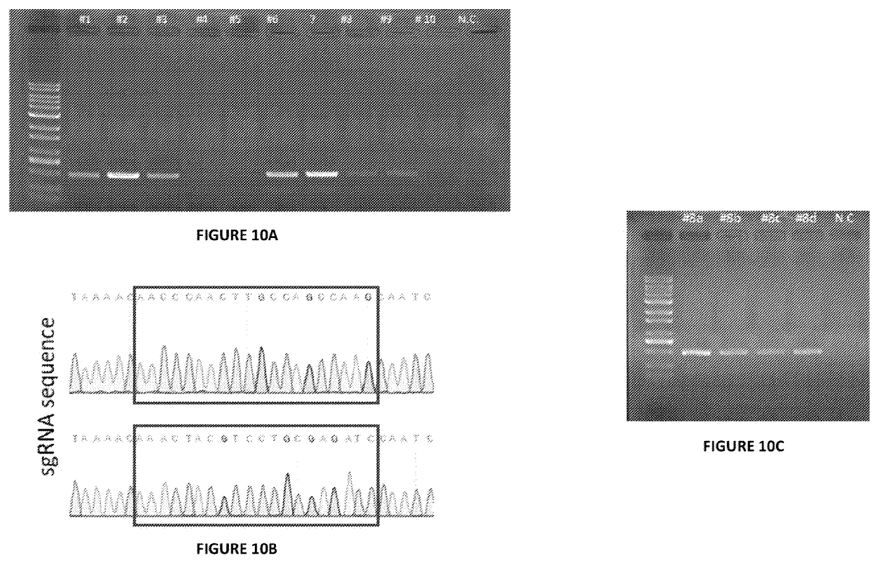

Genotyping transformed tomato T1 plants in greenhouse conditions-30 independent T1 lines were grown in controlled growth rooms with and without NaCl (120 mM) treatment (FIG. 9). We have genotyped the plants to test if they contain the sgRNA cassette. 9 out of the 10 lines in TO showed the expected sgRNA band (FIG. 10A). The sgRNA band was reproducible in T1 plants (FIG. 10C). We further sequenced the sgRNA and confirmed its integration in the plant (FIG. 10B). Importantly, the sgRNA sequence reveals the putative target genes.

### Example 6: Multi-Knock, Multi-Targeted, CRISPR-Based, in Rice

Computational design of a standard library, each construct including a single guide RNA targeting a gene family in rice-A multi-targeted CRISPR library was designed to target the transporter genes in rice, representing a major model crop that is phylogenetically distant from tomato. Together, the rice and tomato systems represent two major flowering-plants lineages (eudicots and monocots). In total, 634 sgRNAs were designed targeting 405 rice transporters. The library was divided into two sub-libraries:
1) ABC+DMT+MFS families: 198 genes targeted by 334 sgRNAs.
2) APC+Chapo+MC+OCCG+OG+VPVHP families: 207 genes targeted by 300 sgRNAs.

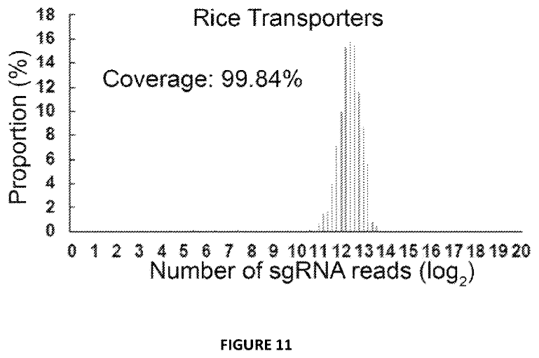

Library synthesis and cloning, including 800 sgRNA constructs—The two rice sgRNA libraries were synthesized and cloned in late 2021. To confirm complete coverage and equal representation of sgRNAs, a deep-sequencing of the libraries was performed. The data showed 99.84% coverage and bell-shaped distribution of equal sgRNA representation in the library (FIG. 11).

Transformation of the library to create 1000 independent rice CRISPR plants-Two transportome-scale sgRNA sub-libraries were transformed into rice to generate 1,000 independent rice lines by tissue culture in the Zhonghua 11 background (outsourced to BioRun, Wuhan, China). Plants were propagated to generate T1 seeds.

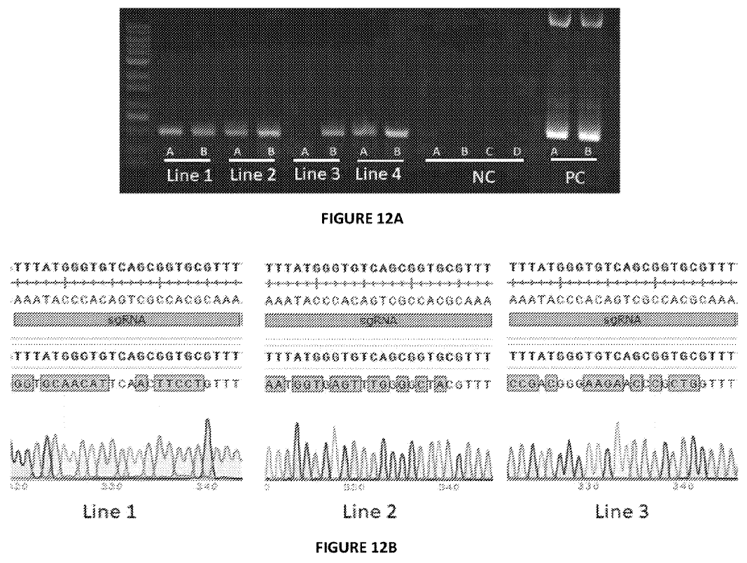

Genotyping transformed rice T1 plants-Independent T1 lines were genotyped to confirm that the plants contain the sgRNA cassette. All lines showed the expected sgRNA band (FIG. 12A). Note that the sgRNA segregates in T1 (e.g., line 3). We further sequenced the sgRNA and confirmed its integration in the plant (FIG. 12B). The sgRNA seq allows to predict the putative target genes.

### Example 7: Multiplexing Multi-Targeted High-Efficiency Genome Editing Library

We have devised a new algorithm for designing the optimal set of sgRNAs for targeting a given gene family to be used within a multiplex CRISPR-Cas genome editing system. In such systems, multiple sgRNAs can be integrated within a single editing vector. The use of multiple sgRNAs, rather than one could allow more efficient editing, either by designing sgRNAs that are each more specific while the entire vector could target a larger fraction of the input gene family. The idea of the algorithm is to scan all potential sgRNA sets and to identify those having the largest editing potential to edit the entire gene set with highest efficiency. While the algorithm is general and can be applied to an sgRNA set of any size, we applied the algorithm for designing a pair of sgRNAs per vector. The algorithm was then coded in Python, incorporated into the CRISPys software, and is available for internal use through the GitHub repository. We have applied the algorithm to 184 gene families in Arabidopsis. In total, 1192 multiplexes were designed with an average of 3.94 genes predicted to be edited per multiplex. The library is now being synthesized to be transformed into plants.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method for identifying multiple members within at least one gene set underlying a phenotype, the method comprising:
(i) clustering coding sequences within genetic data of a plant species to sequence clusters, each cluster representing a gene set;
(ii) producing a CRISPR library comprising a plurality of polynucleotides, wherein each polynucleotide encodes one or more unique sgRNAs, wherein each of the sgRNAs targets a plurality of gene members comprised within the gene set;
(iii) transforming the library into a plurality of plants, thereby producing a plant population wherein each plant of the population comprises at least one sgRNA targeting multiple gene members;
(iv) screening the plant population for at least one selected phenotype;
(v) selecting at least one plant showing the at least one selected phenotype; and
(vi) identifying in the selected plant the at least one sgRNA targeting the multiple-gene members;
thereby identifying said multiple gene members underlying said selected phenotype.

2. The method of claim 1, wherein at least two of the unique sgRNAs target a single gene member.

3. The method of claim 1, wherein at least two of the unique sgRNAs target at least two same gene members out of a plurality of gene members targeted by the at least two unique sgRNAs.

4. The method of claim 3, wherein at least two of the unique sgRNAs target the same plurality of gene members.

5. (canceled)

6. The method of claim 1, wherein the library comprises at least one polynucleotide encoding for two different sgRNAs targeting the same gene members.

7. (canceled)

8. The method of claim 1, wherein the genetic data are RNA sequencing data selected from total RNA-seq and transcriptomics.

9. The method of claim 1, wherein the gene set comprises members of a gene family, and wherein clustering the coding sequences comprises clustering coding sequences encoding polypeptides having at least 30% sequence identity.

10. The method of claim 1, wherein the gene set comprises members of a pathway, and wherein clustering the coding sequences is based on the functional or molecular characteristics of the pathway.

11. The method of claim 1, wherein producing the CRISPR library comprises designing the plurality of sgRNAs following an analysis of the genetic data of the plant.

12. (canceled)

13. (canceled)

14. The method of claim 1, wherein designing the plurality of sgRNA comprises using a computational algorithm determining the probability that a genomic target is cleaved by a given sgRNA.

15. The method of claim 14, wherein the computational algorithm computes all possible sgRNA target sites within the exonic regions on both DNA strands.

16. (canceled)

17. The method of claim 1, wherein said method comprises a step of further sub-grouping the gene set based on their sequence similarity.

18. The method of claim 1, wherein said method comprises producing a plurality of libraries, each library comprising a plurality of polynucleotides, wherein each polynucleotide encoding one or more unique sgRNAs targeting a plurality of gene members comprised within a gene set, wherein each library comprises a different gene set.

19. The method of claim 18, wherein said method comprises producing from 2 to at least 5, at least 10, at least 100, at least 200, at least 500 or more libraries.

20. The method of claim 1, wherein the one or more sgRNAs further comprise at least one adaptor nucleotide, wherein the adaptor nucleotide facilitate amplification of the at least one library.

21. (canceled)

22. (canceled)

23. (canceled)

24. (canceled)

25. The method of claim 1, wherein the one or more unique sgRNAs comprises at least 10, at least 50, at least 100, at least 200, at least 300, at least 400, at least 500, at least 600, at least 700, at least 800, at least 900, at least 1000, or more, sgRNAs.

26. The method of claim 1, wherein the library or the plurality of libraries is transformed into a plurality of plants to form a plurality of transformed plants, each transformed plant expressing at least one sgRNA, each sgRNA targeting multiple members of a gene set.

27. (canceled)

28. The method of claim 1, wherein the selected phenotype is attributed to a genetic manipulation intentionally introduced into the plant population.

29. (canceled)

30. A library for screening multiple members within at least one gene set, the library comprising a plurality of vectors, each vector comprising a polynucleotide encoding one or more unique sgRNAs, wherein each sgRNA is targeted to a plurality of genes, wherein the plurality of genes are members of a gene set.

31. (canceled)

32. (canceled)

33. (canceled)

34. (canceled)

35. (canceled)

36. A construct comprising a plurality of polynucleotides each encoding a unique sgRNA targeting the same gene members within a gene set.

37. (canceled)

38. (canceled)
